(19) United States
(12) Patent Application Publication
(10) Pub. No.: US 2019/0307726 A1
(43) Pub. Date: Oct. 10, 2019

(54) PHARMACEUTICAL FORMULATIONS

(71) Applicant: Molecular Defenses Corporation, New York, NY (US)

(72) Inventors: Arne Holmgren, Stockholm (SE); Harry B. Demopoulos, Scarsdale, NY (US); Kevin Davis, New York, NY (US)

(21) Appl. No.: 16/463,761

(22) PCT Filed: Nov. 23, 2016
(86) PCT No.: PCT/US2016/063616

§ 371 Date: May 23, 2019

Related U.S. Application Data
(60) Provisional application No. 62/260,136, filed on Nov. 25, 2015.

(51) Int. Cl. A61K 31/41; A61K 38/06; A61K 31/428; A61K 9/48

(52) U.S. Cl. CPC A61K 31/41; A61K 9/4825; A61K 31/428; A61K 38/063

(57) ABSTRACT

Disclosed herein is glutathione in conjunction with an isoselenazol or isothiazol derivative, e.g., ebselen or ebsulfur derivative, to treat diabetes, lupus, or other chronic inflammatory disease. The glutathione is preferably provided in a rapid release oral formulation that presents the glutathione for absorption in the first part of the ileum. The isoselenazol or isothiazol derivative is preferably provided in a delayed release formulation to avoid overlapping high enteric concentration. These may be provided within the same unit dosage form.

### FIELD OF THE INVENTION

The present invention relates glutathione and isoselenazol or isothiazol derivatives, their use for treatment of inflammation-mediated disorders, and methods of treatment of such disorders.

### INCORPORATION BY REFERENCE

All publications, patents, and patent applications disclosed herein are incorporated by reference to the same extent as if each individual publication, patent, or patent application was specifically and individually indicated to be incorporated by reference. In the event of a conflict between a term disclosed herein and a term in an incorporated reference, the term herein controls.

### BACKGROUND OF THE INVENTION

Glutathione

The ubiquitous tripeptide L-glutathione (GSH) (gamma-glutamyl-cysteinyl-glycine), is a well-known biological antioxidant, and in fact is believed to be the primary intracellular antioxidant for higher organisms. When oxidized, it forms a dimer (GSSG), which may be recycled in organs having glutathione reductase. Glutathione may be transported through membranes by the sodium-dependent glutamate pump. Tanuguchi, N., et al. Eds., Glutathione Centennial, Academic Press, New York (1989). The properties of glutathione (“GSH”) derive from just a few central facts including the molecular configuration of L-gamma glutamylcysteinyl glycine, its controlled reactivity, its ability to maintain a physiologically favorable Redox potential, its antioxidant properties in all subcellular compartments, the existence of avid glutathione transporters on cell membranes and mitochondria and the fact that these properties are supported by enzymes: (i) those that synthesize GSH; (ii) enzymes that amplify particular properties, such as GSH peroxidases and S-transferases and; (iii) enzymes that restore GSH after it has been used, GSH reductase. The properties of glutathione have been categorized into four groups, but can be organized differently.

GSH is known to function directly or indirectly in many important biological phenomena, including the synthesis of proteins and DNA, transport, enzyme activity, metabolism, and protection of cells from free-radical mediated damage. GSH is one of the primary cellular antioxidants responsible for maintaining the proper oxidation state within the body. GSH is synthesized by most cells, and is also supplied in the diet. GSH has been shown to recycle oxidized biomolecules back to their active, reduced forms.

Because of the existing mechanisms for controlling interconversion of reduced and oxidized glutathione, an alteration of the level of reduced glutathione (GSH), e.g., by administration of GSH to an organism will tend to shift the cells of the organism to a more reduced redox potential. Likewise, subjecting the organism to oxidative stress or free radicals will tend to shift the cells to a more oxidized potential. It is well known that certain cellular processes are responsive to redox potential.

Reduced glutathione (GSH) is, in the human adult, produced from oxidized glutathione (GSSG) primarily by the liver, and to a smaller extent, by the skeletal muscle, red blood cells, and white cells. About 80% of the 8-10 grams glutathione produced daily is produced by the liver and distributed through the blood stream to the other tissues. A deficiency of glutathione in cells may lead to excess free radicals, which cause macromolecular breakdown, lipid peroxidation, buildup of toxins, and ultimately cell death. Because of the importance of glutathione in preventing this cellular oxidation, glutathione is continuously supplied to the tissues. However, under certain conditions, the normal, physiologic supplies of glutathione are insufficient, distribution inadequate or local oxidative demands too high to prevent cellular oxidation. Under certain conditions, the production of and demand for glutathione are mismatched, leading to insufficient levels on an organismal level. In other cases, certain tissues or biological processes consume glutathione so that the intracellular levels are suppressed. In either case, by increasing the serum levels of glutathione, increased amounts may be directed into the cells. In facilitated transport systems for cellular uptake, the concentration gradient which drives uptake is increased.

As with all nutrients, eating or orally ingesting the nutrient would generally be considered a desired method for increase body levels thereof. Glutathione is relatively unstable in alkaline or oxidative environments, and is not absorbed by the stomach. It is believed that glutathione is absorbed, after oral administration, if at all, in the latter half of the duodenum and the beginning of the jejunum. It was also believed that orally administered glutathione would tend to be degraded in the stomach, and that it is particularly degraded under alkaline conditions by desulfurases and peptidases present in the duodenum.

Pure glutathione forms a flaky powder that retains a static electrical charge, due to triboelectric effects, making processing and formulation difficult. The powder particles may also have an electrostatic polarization, which is akin to an electret. Glutathione is a strong reducing agent, so that autooxidation occurs in the presence of oxygen or other oxidizing agents. U.S. Pat. No. 5,204,114 provides a method of manufacturing glutathione tablets and capsules by the use of crystalline ascorbic acid as an additive to reduce triboelectric effects which interfere with high speed equipment and maintaining glutathione in a reduced state. A certain crystalline ascorbic acid is, in turn, disclosed in U.S. Pat. No. 4,454,125, which is useful as a lubricating agent for machinery. Ascorbic acid has the advantage that it is well tolerated, antioxidant, and reduces the net static charge on the glutathione.

A number of disease states have been specifically associated with reductions in glutathione levels. Depressed glutathione levels, either locally in particular organs, or systemically, have been associated with a number of clinically defined diseases and disease states. These include HIV/AIDS, diabetes, systemic lupus erythematosus, and macular degeneration, all of which progress because of excessive free radical reactions and insufficient GSH. Other chronic conditions may also be associated with GSH deficiency, including heart failure and coronary artery restenosis post angioplasty.

Systemic lupus erythematosus (SLE) is reported to be characterized by imbalance redox state and increased apoptosis. Shah, Dilip, Sangita Sah, and Swapan K. Nath. “Interaction between glutathione and apoptosis in systemic lupus erythematosus.” Autoimmunity reviews 12.7 (2013): 741-751. The activation, proliferation and cell death of lymphocytes are dependent on intracellular levels of glutathione and controlled production of reactive oxygen species (ROS). Changes in the intracellular redox environment of cells, through oxygen-derived free radical production known as oxidative stress, have been reported to be critical for cellular immune dysfunction, activation of apoptotic enzymes and apoptosis. The shift in the cellular GSH-to-GSSG redox balance in favor of the oxidized species, GSSG, constitutes an important signal that can decide the fate of the abnormal apoptosis in the disease.

Clinical and pre-clinical studies have demonstrated the linkage between a range of free radical disorders and insufficient GSH levels. Newly published data implies that diabetic complications are the result of hyperglycemic episodes that promote glycation of cellular enzymes and thereby inactivate GSH synthetic pathways. The result is GSH deficiency in diabetics, which may explain the prevalence of cataracts, hypertension, occlusive atherosclerosis, and susceptibility to infections in these patients.

GSH therapy: (1) assures glutathione availability to support TH1 immunologic responses needed to recover from smallpox; (2) slows activation and over-expression of NFκB and inflammatory cascades that cause cumulative tissue toxicities; and (3) biochemically neutralizes reactive intermediates that otherwise cause cellular and tissue toxicities. Consistently normal intracellular concentrations of GSH help maintain the balance of T Helper 1 and 2 (TH1 and TH2) immunologic response patterns. When GSH is continuously lost, restorative GSH therapy rapidly up-regulates TH1, enhancing Interferon γ and cell mediated immunity required for recovery, while down-regulating IL-4, a disadvantageous TH2 cytokine, when over expressed during acute viral infections. This beneficial effect of GSH, required for recovery responses, has been demonstrated against other dangerous viruses, including pox viruses. Consistently normal intracellular concentrations of glutathione also sets a high reduction oxidation potential within cells, that slows activation and over-expression of NFκB, TNFα, IL-1β, adhesion molecules, cyclo-oxygenase-2, matrix metalloproteinases and inflammatory cascades. This mechanism has also been demonstrated against other dangerous viruses, including pox viruses. The ability to help control such reactions indicates further, potential uses of GSH in counter terrorism.

Biochemical neutralizing reactions: (a) GSH neutralizes reactive oxygen and reactive nitrogen species (ROS and RNS) continuously produced during viral infections that otherwise damage cell membrane lipids, proteins, and nucleic acids, and result in cellular and tissue toxicities; (b) GSH protects mitochondria against hydrogen peroxide, bioenergetic failure, and exaggerated, apoptotic processes that add to cumulative tissue toxicities; (c) Consistently normal intracellular concentrations of GSH help control non enzymatic and enzymatic oxidations of arachidonic acid. Otherwise these cause tissue-disrupting excesses of reactive intermediates from lipid hydroperoxides (alkoxy radicals, LO., and hydroxyls, .OH).

High normal GSH concentrations in dendritic cells, macrophages and lymphocytes rapidly up-regulate T helper 1 (Th1) response patterns (ex. IL-12, Interferon gamma, and specific cell mediated immunity), required for recovery from viral infections and down-regulate T helper 2 (Th2) response patterns (ex. IL-4, IL-10, and humoral immunity). Th1 and Th2 response patterns must be balanced, timed, and controlled. Thiols have long been recognized as important in Th1/Th2 response patterns. Glutathione concentrations in pivotal cells such as monocytes/macrophages, dendritic cells, and lymphocytes are vulnerable and decline rapidly in response to alcohol, toxins, oxidative stresses, physical/emotional stress, infections, trauma, burns, and non-bacterial and bacterial sepsis. The Th1/Th2 balance shifts to Th2 predominance as a result, making recoveries difficult.

Glutathione (GSH) maintains the Redox Potential, i.e. the Reducing vs. the Oxidizing Potential [GSH]/[GSSG], within cells. This ratio is in the range of 500. The normal [GSH] in cells is 5-10 mM. GSH is a major determinant of the Reduction Oxidation Potential in the cell and is protectively involved in diverse cell activities, including “ . . . Control of cell cycle progression in human natural killer cells . . . ”, and defensive responses to infections, to chemical exposures, and to other detrimental factors such as diesel exhaust particles, aging, diabetes, and photo-oxidative retinal damage. As noted by the CDC, “ . . . physiologic host factors make the difference in a case (of smallpox) and how severe it will be . . . ”. The effects of glutathione concentrations, [GSH], on Redox and the consequent effects on specific entities such as the NFκB family, TNFα, cytokines, COX-2 and adhesion molecules, provide substance for the term, “ . . . host factors . . . ”, and also provide direction for additional therapeutics, for example, raising [GSH] and simultaneously protecting the patient from chemical toxins and other factors detrimental to [GSH], as cited previously and below.

Biochemical Evolution proceeded towards a stable/controllable range of pH, pO₂, osmolarity, and [Na⁺]/[K⁺]; so too has this process led to a stable/controllable Redox. When the concentration of GSH is high, Redox is high. Then, it can control and slow the excess activation of the NFκB family, “ . . . oxidant-sensitive transcription regulator(s) . . . ”, of proinflammatory cytokines; COX-2; adhesion molecules; and TNFα and IL-1β that cause secondary cascades. A significant decrease in GSH results in a decline in Redox and activation of the NFκB family and the other factors.

Use of GSH to Treat Diabetes Mellitus Obesity is characterized by inflammation. Kathryn E. Wellen and Gokhan S. Hotamisligil, “Inflammation, stress, and diabetes”, J Clin Invest. 2005 May 2; 115(5): 1111-1119. doi: 10.1172/JC1200525102. PMCID: PMC1087185. The first molecular link between inflammation and obesity, TNF-α, was identified when it was discovered that this inflammatory cytokine is overexpressed in the adipose tissues of rodent models of obesity. As is the case in mice, TNF-α is overproduced in the adipose as well as muscle tissues of obese humans. Administration of recombinant TNF-α to cultured cells or to whole animals impairs insulin action, and obese mice lacking functional TNF-α or TNF receptors have improved insulin sensitivity compared with wild-type counterparts. Thus, particularly in experimental models, it is clear that overproduction of TNF-α in adipose tissue is an important feature of obesity and contributes significantly to insulin resistance. Obesity is characterized by a broad inflammatory response and that many inflammatory mediators exhibit patterns of expression and/or impact insulin action in a manner similar to that of TNF-α during obesity, in animals ranging from mice and cats to humans. Transcriptional profiling studies have revealed that inflammatory and stress-response genes are among the most abundantly regulated gene sets in adipose tissue of obese animals. In addition to inflammatory cytokines regulating metabolic homeostasis, molecules that are typical of adipocytes, with well-established metabolic functions, can regulate the immune response. Leptin is one such hormone that plays important roles in both adaptive and innate immunity, and both mice and humans lacking leptin function exhibit impaired immunity. Indeed, reduced leptin levels may be responsible, at least in part, for immunosuppression associated with starvation, as leptin administration has been shown to reverse the immunosuppression of mice starved for 48 hours. Adiponectin, resistin, and visfatin are also examples of molecules with immunological activity that are produced in adipocytes. Finally, lipids themselves also participate in the coordinate regulation of inflammation and metabolism. Elevated plasma lipid levels are characteristic of obesity, infection, and other inflammatory states. Hyperlipidemia in obesity is responsible in part for inducing peripheral tissue insulin resistance and dyslipidemia and contributes to the development of atherosclerosis. It is interesting to note that metabolic changes characteristic of the acute-phase response are also proatherogenic; thus, altered lipid metabolism that is beneficial in the short term in fighting against infection is harmful if maintained chronically. The critical importance of bioactive lipids is also evident in their regulation of lipid-targeted signaling pathways through fatty acid-binding proteins (FABPs) and nuclear receptors.

The high level of coordination of inflammatory and metabolic pathways is highlighted by the overlapping biology and function of macrophages and adipocytes in obesity. Obesity is associated with a state of chronic, low-grade inflammation, particularly in white adipose tissue. Insulin affects cells through binding to its receptor on the surface of insulin-responsive cells. The stimulated insulin receptor phosphorylates itself and several substrates, including members of the insulin receptor substrate (IRS) family, thus initiating downstream signaling events. The inhibition of signaling downstream of the insulin receptor is a primary mechanism through which inflammatory signaling leads to insulin resistance. Exposure of cells to TNF-α or elevated levels of free fatty acids stimulates inhibitory phosphorylation of serine residues of IRS-1. This phosphorylation reduces both tyrosine phosphorylation of IRS-1 in response to insulin and the ability of IRS-1 to associate with the insulin receptor and thereby inhibits downstream signaling and insulin action. Inflammatory signaling pathways can also become activated by metabolic stresses originating from inside the cell as well as by extracellular signaling molecules. Obesity overloads the functional capacity of the ER and that this ER stress leads to the activation of inflammatory signaling pathways and thus contributes to insulin resistance. Additionally, increased glucose metabolism can lead to a rise in mitochondrial production of ROS. ROS production is elevated in obesity, which causes enhanced activation of inflammatory pathways.

Several serine/threonine kinases are activated by inflammatory or stressful stimuli and contribute to inhibition of insulin signaling, including JNK, inhibitor of NFκB kinase (IKK), and PKC-θ). Again, the activation of these kinases in obesity highlights the overlap of metabolic and immune pathways; these are the same kinases, particularly IKK and JNK, that are activated in the innate immune response by Toll-like receptor (TLR) signaling in response to LPS, peptidoglycan, double-stranded RNA, and other microbial products. Hence it is likely that components of TLR signaling pathways will also exhibit strong metabolic activities. Two other inflammatory kinases that play a large role in counteracting insulin action, particularly in response to lipid metabolites, are IKK and PKC-θ. Lipid infusion has been demonstrated to lead to a rise in levels of intracellular fatty acid metabolites, such as diacylglycerol (DAG) and fatty acyl CoAs. This rise is correlated with activation of PKC-θ and increased Ser307 phosphorylation of IRS-1. PKC-θ may impair insulin action by activation of another serine/threonine kinase, IKKβ, or JNK. Other PKC isoforms have also been reported to be activated by lipids and may also participate in inhibition of insulin signaling. IKKβ can impact on insulin signaling through at least 2 pathways. First, it can directly phosphorylate IRS-1 on serine residues. Second, it can phosphorylate inhibitor of NFκB (IκB), thus activating NFκB, a transcription factor that, among other targets, stimulates production of multiple inflammatory mediators, including TNF-α and IL-6. Mice heterozygous for IKKβ are partially protected against insulin resistance due to lipid infusion, high-fat diet, or genetic obesity. Moreover, inhibition of IKKβ in human diabetics by high-dose aspirin treatment also improves insulin signaling, although at this dose, it is not clear whether other kinases are also affected. Recent studies have also begun to tease out the importance of IKK in individual tissues or cell types to the development of insulin resistance. Activation of IKK in liver and myeloid cells appears to contribute to obesity-induced insulin resistance, though this pathway may not be as important in muscle. In addition to serine/threonine kinase cascades, other pathways contribute to inflammation-induced insulin resistance. For example, at least 3 members of the SOCS family, SOCS1, -3, and -6, have been implicated in cytokine-mediated inhibition of insulin signaling. These molecules appear to inhibit insulin signaling either by interfering with IRS-1 and IRS-2 tyrosine phosphorylation or by targeting IRS-1 and IRS-2 for proteosomal degradation. SOCS3 has also been demonstrated to regulate central leptin action, and both whole body reduction in SOCS3 expression (SOCS3+/−) and neural SOCS3 disruption result in resistance to high-fat diet-induced obesity and insulin resistance.

Inflammatory cytokine stimulation can also lead to induction of iNOS. Overproduction of nitric oxide also appears to contribute to impairment of both muscle cell insulin action and p cell function in obesity. Deletion of iNOS prevents impairment of insulin signaling in muscle caused by a high-fat diet. Thus, induction of SOCS proteins and iNOS represent 2 additional and potentially important mechanisms that contribute to cytokine-mediated insulin resistance. It is likely that additional mechanisms linking inflammation with insulin resistance remain to be uncovered.

The role of lipids in metabolic disease is complex. As discussed above, hyperlipidemia leads to increased uptake of fatty acids by muscle cells and production of fatty acid metabolites that stimulate inflammatory cascades and inhibit insulin signaling. On the other hand, intracellular lipids can also be antiinflammatory. Ligands of the liver X receptor (LXR) and PPAR families of nuclear hormone receptors are oxysterols and fatty acids, respectively, and activation of these transcription factors inhibits inflammatory gene expression in macrophages and adipocytes, in large part through suppression of NFκB. LXR function is also regulated by innate immune pathways. Signaling from TLRs inhibits LXR activity in macrophages, causing enhanced cholesterol accumulation and accounting, at least in part, for the proatherogenic effects of infection. Indeed, lack of MyD88, a critical mediator of TLR signaling, reduces atherosclerosis in apoE−/− mice. Interestingly, despite the inhibitory effects of TLR signaling on LXR cholesterol metabolism, LXR appears to be necessary for the complete response of macrophages to infection. In the absence of LXR, macrophages undergo accelerated apoptosis and are thus unable to appropriately respond to infection. Unliganded PPARδ also seems to have proinflammatory functions, mediated at least in part through its association with the transcriptional repressor B cell lymphoma 6 (BCL-6). The activity of these lipid ligands is influenced by cytosolic FABPs. Animals lacking the adipocyte/macrophage FABPs ap2 and mall are strongly protected against type 2 diabetes and atherosclerosis, a phenotype reminiscent of that of thiazolidinedione-treated (TZD-treated) mice and humans. One mechanism for this phenotype is potentially related to the availability of endogenous ligands for these receptors that stimulate storage of lipids in adipocytes and suppress inflammatory pathways in macrophages. In general, it appears that location in the body, the composition of the surrounding cellular environment, and coupling to target signaling pathways are critical for determining whether lipids promote or suppress inflammation and insulin resistance. Accumulation of cholesterol in macrophages promotes atherosclerosis and of lipid in muscle and liver promotes insulin resistance, while, as seen in TZD-treated and FABP-deficient mice, if lipids are forced to remain in adipose tissue, insulin resistance in the context of obesity can be reduced. Thus, lipids and their targets clearly play both metabolic and inflammatory roles; however, the functions that they assume are dependent on multiple factors.

In corroboration of genetic evidence in mice that loss of inflammatory mediators or signaling molecules prevents insulin resistance, pharmacological targeting of inflammatory pathways also improves insulin action. Effective treatment has been demonstrated both with inhibitors of inflammatory kinases and with agonists of relevant transcription factors. As discussed above, salicylates promote insulin signaling by inhibiting inflammatory kinase cascades within the cell. Through inhibition of IKK and possibly other kinases, salicylates are able to improve glucose metabolism in both obese mice and diabetic humans. Targeting of JNK using a synthetic inhibitor and/or an inhibitory peptide has been demonstrated to improve insulin action in obese mice and reduce atherosclerosis in the apoE-deficient rodent model. These results directly demonstrate the therapeutic potential of JNK inhibitors in diabetes.

One mechanism that may be of importance in the activation of inflammatory pathways associated with obesity is ER stress. Obesity generates conditions that increase the demand on the ER. This is particularly the case for adipose tissue, which undergoes severe changes in tissue architecture, increases in protein and lipid synthesis, and perturbations in intracellular nutrient and energy fluxes. In both cultured cells and whole animals, ER stress leads to activation of JNK and thus contributes to insulin resistance. Interestingly, ER stress also activates IKK and thus may represent a common mechanism for the activation of these 2 important signaling pathways. A second mechanism that may be relevant in the initiation of inflammation in obesity is oxidative stress. Due to increased delivery of glucose to adipose tissue, endothelial cells in the fat pad may take up increasing amounts of glucose through their constitutive glucose transporters. Increased glucose uptake by endothelial cells in hyperglycemic conditions causes excess production of ROS in mitochondria, which inflicts oxidative damage and activates inflammatory signaling cascades inside endothelial cells. Endothelial injury in the adipose tissue might attract inflammatory cells such as macrophages to this site and further exacerbate the local inflammation. Hyperglycemia also stimulates ROS production in adipocytes, which leads to increased production of proinflammatory cytokines. Finally, in addition to diabetes and cardiovascular disease, inflammation is also known to be important for linking obesity to airway inflammation and asthma, fatty liver disease, and possibly cancer and other pathologies.

Diabetes mellitus is found in two forms, childhood or autoimmune (type I, IDDM) and late-onset or non-insulin dependent (type II, NIDDM), associated with obesity. The former constitute about 30% and the remainder represent the bulk of cases seen. Onset is generally sudden for Type I, and insidious for Type II. Symptoms include excessive urination, hunger and thirst with a slow steady loss of weight in the first form. Obesity is often associated with the second form and has been thought to be a causal factor in susceptible individuals. Blood sugar is often high and there is frequent spilling of sugar in the urine. If the condition goes untreated, the victim may develop ketoacidosis with a foul-smelling breath similar to someone who has been drinking alcohol. The immediate medical complications of untreated diabetes can include nervous system symptoms, and even diabetic coma. Because of the continuous and pernicious occurrence of hyperglucosemia (very high blood sugar levels), a non-enzymatic chemical reaction occurs called glycation. Since glycation occurs far more frequently inside cells, the inactivation of essential enzyme proteins happens almost continually. One of the most critical enzymes, γ-glutamyl-cysteine synthetase, is glycated and readily inactivated. This enzyme is the crucial step in the biosynthesis of glutathione in the liver. The net result of this particular glycation is a deficiency in the production of GSH in diabetics. Normally, adults produce 8-10 grams every 24 hours, and it is rapidly oxidized by the cells. GSH is in high demand throughout the body for multiple, essential functions, for example, within all mitochondria, to produce chemical energy called ATP. Brain cells, heart cells, and others simply will not function well and can be destroyed through apoptosis.

GSH is the major antioxidant in the human body and the only one we are able to synthesize, de novo. It is also the most common small molecular weight thiol in both plants and animals. Without GSH, the immune system cannot function, and the central and peripheral nervous systems become aberrant and then cease to function. Because of the dependence on GSH as the carrier of nitric oxide, a vasodilator responsible for control of vascular tone, the cardiovascular system does not function well and eventually fails. Since all epithelial cells seem to require GSH, the intestinal lining cells don't function properly and valuable micronutrients are lost, nutrition is compromised, and microbes are given portals of entry to cause infections.

The use of GSH precursors cannot help to control the GSH deficiency due to the destruction of the rate-limiting enzyme by glycation. As GSH deficiency becomes more profound, the well-known sequellae of diabetes progress in severity. The complications described below are essentially due to runaway free radical damage since the available GSH supplies in diabetics are insufficient.

Reducing sugars are known to interact with free amino groups in proteins, lipids, and nucleic acids to form Amadori product and produce reactive oxygen species through the glycation reaction. Under diabetic conditions, glucose level is elevated and the glycated proteins increased. Cu,Zn-SOD has been shown to be glycated and inactivated under diabetic conditions and that ROS produced from the Amadori product caused site-specific fragmentation of Cu,Zn-SOD. Fructose, which is produced through polyol pathway, has stronger glycating capacity than glucose because the physiologic proportion of the linear form is higher than that of cyclized form. Fructose, as well as ribose, can bring about apoptosis in pancreatic p islet cell line. Levels of intracellular peroxides, protein carbonyls, and malondialdehyde are increased in the presence of fructose. In addition, methylglyoxal and 3-deoxyglucosone have also been shown to induce apoptotic cell death. 3-Deoxyglucosone, a 2-oxoaldehyde, is produced through the degradation of Amadori compounds. Both compounds are elevated during hyperglycemia and accelerate the glycation reaction. These compounds are toxic to cells, due to their high reactivity, and a scavenging system with NADPH-dependent reducing activity exists, including aldehyde reductase. Junichi Fujii and Naoyuki Taniguchi, Dysfunction of Redox System by Reactive Oxygen Species, Nitric Oxide and the Glycation Reaction: A Possible Mechanism for Apoptotic Cell Death (Poster), Proceedings of 3rd Internet World Congress on Biomedical Sciences, 1996.12.9-20 Riken, Tsukuba, Japan.

Cell-cell adhesion is critical in generation of effective immune responses and is dependent upon the expression of a variety of cell surface receptors. Intercellular adhesion molecule-1 (ICAM-1; CD54) and vascular cell adhesion molecule (VCAM-1; CD 106) are inducible cell surface glycoproteins. The expression of these surface proteins are known to be induced in response to activators such as cytokines (TNF-α, IL-1 α & β), PMA, lipopolysaccharide and oxidants. The ligands for ICAM-1 and VCAM-1 on lymphocyte are LFA-1 (CD11a/CD18) and VLA-4, respectively. The inappropriate or abnormal sequestration of leukocytes at specific sites is a central component in the development of a variety of autoimmune diseases and pathologic inflammatory disorders. Focal expression of ICAM-1 have been reported in arterial endothelium overlying early foam cell lesions in both dietary and genetic models of atherosclerosis in rabbits. A role of VCAM-1 in the progression of coronary lesions has also been suggested. Loss or gain of cell surface molecules is thought to determine the mobilization, emigration and invasiveness of epithelial cancer cells. Monocytes from patients with diabetes mellitus are known to have increased adhesion to endothelial cells in culture. Regulation of adhesion molecule expression and function by reactive oxygen species via specific redox sensitive mechanisms have been reported. Antioxidants can block induced adhesion molecule expression and cell-cell adhesion. Sashwati Roy and Chandan K. Sen, Adhesion Molecules And Cell-Cell Adhesion, http://packer.berkeley.edu/research/Cell/adhes.

The diabetic will become more susceptible to infections because the immune system approaches collapse when GSH levels fall, analogous to certain defects seen in HIV/AIDS. Peripheral vasculature becomes compromised and blood supply to the extremities is severely diminished because GSH is not available in sufficient amounts to stabilize the nitric oxide (.NO) to effectively exert its vascular dilation (relaxation) property. Gangrene is a common sequel and successive amputations are often the result in later years. Peripheral neuropathies, the loss of sensation commonly of the feet and lower extremities develop, often followed by aberrant sensations like burning or itching, which can't be controlled. Retinopathy and nephropathy are later events that are actually due to microangiopathy, excessive budding and growth of new blood vessels and capillaries, which often will bleed due to weakness of the new vessel walls. This bleeding causes damage to the retina and kidneys with resulting blindness and renal shutdown, the latter results in required dialysis. Cataracts occur with increasing frequency as the GSH deficiency deepens. Large and medium sized arteries become sites of accelerated, severe atherosclerosis, with myocardial infarcts at early ages, and of a more severe degree. If diabetics go into heart failure, their mortality rates at one year later are far greater than in non-diabetics. Further, if coronary angioplasty is used to treat their severe atherosclerosis, diabetics are much more likely to have renarrowing of cardiac vessels, termed restenosis. The above complications are due, in large measure, to GSH deficiency and ongoing free radical reactions. These sequellae frequently and eventually occur despite the use of insulin injections daily that lower blood sugar levels. Good control of blood sugar levels is difficult for the majority of diabetics.

Thioredoxin (TRX) is a pleiotropic cellular factor which has thiol-mediated redox activity and plays important roles in regulation of cellular processes, including gene expression. TRX exists either in a reduced, or oxidized form and participates in redox reactions through the reversible oxidation of this active center dithiol. Activity of a number of transcription factors is post-translationally altered by redox modification(s) of specific cysteine residue(s). One such factor is NFκB, whose DNA-binding activity is altered by TRX treatment in vitro. The DNA-binding activity of AP-1 is modified by a DNA repair enzyme, Redox Factor-1 (Ref-1). Ref-1 activity is in turn modified by various redox-active compounds, including TRX. TRX translocates from the cytoplasm into the nucleus in response to PMA treatment to associate directly with Ref-1 and modulates not only the DNA-binding but also the transcriptional activity of the AP-1 molecule.

Human thioredoxin (hTRX) has thus been shown to be an important redox regulator in those biological processes. hTRX can function directly by interacting with the target molecules such as NFκB transcription factor, or indirectly via another redox protein known as redox factor 1 (Ref-1). See, Structural Basis Of Thioredoxin-Mediated Redox-Regulation, Qin et al, (poster), Proceedings of 3rd Internet World Congress on Biomedical Sciences, 1996.12.9-20 Riken, Tsukuba, Japan. Cellular redox status modulates various aspects of cellular events including proliferation and apoptosis. TRX is a small (13 kDa), ubiquitous protein with two redox-active half-cysteine residues in an active center, -Trp-Cys-Gly-Pro-Cys-, and is also known as adult T-cell leukemia-derived factor (ADF) involved in HTLV-I leukemogenesis. The pathway for the reduction of a protein disulfide by TRX entails nucleophilic attack by one of the active-site sulfhydryls to form a protein-protein disulfide followed by intramolecular displacement of the reduced target proteins with concomitant formation of oxidized TRX. Besides the activity as an autocrine growth factor for HTLV-I-infected T cells and Epstein-Barr virus-transformed lymphocytes, numerous studies have shown the importance of ADF/TRX as a cellular reducing catalyst in human physiology.

In vitro and in vivo experiments showed that TRX augmented the DNA-binding and transcriptional activities of the p50 subunit of NFκB by reducing Cys 62 of p50. Direct physical association of TRX and an oligopeptide from NFκB p50 has been revealed by NMR study in vitro. Redox regulation of Jun and Fos molecules has also been implicated. Various antioxidants strongly activate the DNA-binding and transactivation abilities of AP-1 complex. TRX enhances the DNA-binding activity of Jun and Fos, in a process which requires other molecules, such as redox factor-1 (Ref-1).

NFκB regulates expression of a wide variety of cellular and viral genes. These genes include cytokines such as IL-2, IL-6, IL-8, GM-CSF and TNF, cell adhesion molecules such as ICAM-1 and E-selectin, inducible nitric oxidase synthase (iNOS) and viruses such as human immunodeficiency virus (HIV) and cytomegalovirus. NFκB is considered to be causally involved in the currently intractable diseases such as acquired immunodeficiency syndrome (AIDS), hematogenic cancer cell metastasis and rheumatoid arthritis (RA). Although the genes induced by NFκB are variable according to the context of cell lineage and are also under the control of the other transcription factors, NFκB plays a major role in regulation of these genes and thus contributes a great deal to the pathogenesis. Therefore, biochemical intervention of NFκB should conceivably interfere the pathogenic process and would be effective for the treatment. NFκB consists of two subunit molecules, p65 and p50, and usually exists as a molecular complex with an inhibitory molecule, IκB, in the cytosol. Upon stimulation of the cells such as by proinflammatory cytokines, IL-1 and TNF, IκB is dissociated and NFκB is translocated to the nucleus and activates expression of target genes. Thus activity of NFκB itself is regulated by the upstream regulatory mechanism. Not much is known about the upstream signaling cascade. However, there are at least two independent steps in the NFκB activation cascade: kinase pathways and redox-signaling pathway. These two distinct pathways are involved in the NFκB activation cascade in a coordinate fashion, which may contribute to a fine tune, as well as fail-safe, regulation of NFκB activity. At least two distinct types of kinase pathways are known to be involved in NFκB activation: NFκB kinase and IκB kinase. NFκB kinase is a 43 kD serine kinase, associated with NFκB. This kinase phosphorylates both subunits of NFκB and dissociates it from IκB. There is another kinase or kinases that is known to phosphorylate IκB. Consistent with these findings, NFκB was shown to be phosphorylated in some cell lines and IκB was phosphorylated in others in response to stimulation with TNF or IL-1. In most of the cases, NFκB dissociation by kinase cascade is a primary step of NFκB activation.

After dissociation from IκB, however, NFκB must go through the redox regulation by cellular reducing catalyst, thioredoxin (TRX). TRX is known to participate in redox reactions through reversible oxidation of its active center dithiol to a disulfide. Human TRX has been initially identified as a factor responsible for induction of the A subunit of interleukin-2 receptor which is now known to be under the control of NFκB. It is known that NFκB cannot bind to the KB DNA sequence of the target genes until it is reduced. NFκB appears to have a novel DNA-binding structure called beta-barrel, a group of beta sheets stretching toward the target DNA. There is a loop in the tip of the beta barrel structure that intercalates with the nucleotide bases and is considered to make a direct contact with the DNA. This DNA-binding loop contains the cysteine 62 residue of NFκB that is likely the target of redox regulation as a proton donor from TRX. A boot-shaped hollow on the surface of TRX containing the redox-active cysteines could stably recognize the DNA-binding loop of p50 and is likely to reduce the oxidized cysteine by donating protons in a structure-dependent way. Therefore, the reduction of NFκB by TRX is considered to be specific. Pretreatment of cells with antioxidants such as N-acetyl-cysteine (NAC) or a-lipoic acid blocks NFκB. NAC can also block the induction of TRX. Therefore, anti-NFκB actions of antioxidants are considered to be two-fold: 1) blocking the signaling immediately downstream of the signal elicitation, and 2) suppression of induction of the redox effector TRX. It is noted that, in mammals without chronic diseases, such as HIV infection, diabetes, etc., which might impair physiologic glutathione metabolism, a strategy for the pharmaceutical administration of other antioxidants which improve glutathione metabolism or compounds which are themselves appropriate antioxidants may be employed. It is noted that NAC has been shown to have certain neurological toxicity in chronic administration, and therefore this compound is likely inappropriate. On the other hand, lipoic acid may be an advantageous antioxidant alone, or in combination with glutathione. Because of the sensitivity of glutathione oral administration to the particular method of administration, a-lipoic acid may have to be administered separately.

The intracellular redox cascade involves successive reduction of oxygen by addition of four electrons and redox regulation of a target protein. Among these ROI hydrogen peroxide has a longest half-life and is considered to be a mediator of oxidative signal. On the other hand, cellular reducing system such as TRX counteracts the action of hydrogen peroxide. The intensity of the oxidative signal may be modulated by the internal GSH level. Similarly, total GSH/GSSG content may influence the responsiveness of the cellular redox signaling. Therefore, intracellular cysteine required to produce GSH. Reactive oxygen species (ROS) are implicated in the pathogenesis of a wide variety of human diseases. Recent evidence suggests that at moderately high concentrations, certain forms of ROS such as H₂O₂ may act as signal transduction messengers. At least two well-defined transcription factors, nuclear factor (NFκB) and activator protein (AP) -1 have been identified to be regulated by the intracellular redox state. R. Schreck, P. Rieber & P. A. Baeuerle, Reactive oxygen intermediates as apparently widely used messengers in the activation of the NF-κB transcription factor and HIV-1. EMBO J 10: 2247-2258 (1991). Binding sites of the redox-regulated transcription factors NFκB and AP-1 are located in the promoter region of a large variety of genes that are directly involved in the pathogenesis of diseases, e.g., AIDS, cancer, atherosclerosis and diabetic complications. Biochemical and clinical studies have indicated that antioxidant therapy may be useful in the treatment of disease. Critical steps in the signal transduction cascade are sensitive to oxidants and antioxidants. Many basic events of cell regulation such as protein phosphorylation and binding of transcription factors to consensus sites on DNA are driven by physiological oxidant-antioxidant homeostasis, especially by the thiol-disulfide balance. Endogenous glutathione and thioredoxin systems may therefore be considered to be effective regulators of redox-sensitive gene expression. By controlling redox cascades by using antioxidants, for example, treatments for several diseases may be possible, such as hematogenic cancer cell metastasis and AIDS. See, Sen, C. K., Packer, L. Antioxidant and redox regulation of gene transcription. FASEB J. 10, 709-720 (1996). Membrane receptors and transporters, including, for example, the insulin receptor and receptors for certain neurotransmitters, are regulated by the redox state of the cell. A very large number of enzymes are also regulated by the cell's redox state. A partial list of proteins whose function is regulated by oxidation-reduction is presented in Table 1. Babior, B. M. (1997). “Superoxide: a two-edged sword”, Braz. J. Med. Biol. Res., 30, 141-155.

Lupus and Autoimmune Inflammatory Diseases

Systemic lupus erythematosus, often abbreviated as SLE or lupus, is a systemic autoimmune disease (or autoimmune connective tissue disease) in which the body's immune system mistakenly attacks healthy tissue. en.wikipedia.org/wiki/Systemic_lupus_erythematosus, expressly incorporated herein by reference, including cited references therein. Lupus is characterized by the presence of antibodies against a person's own proteins; these are most commonly anti-nuclear antibodies, which are found in nearly all cases. These antibodies lead to inflammation. There is no cure for SLE. It is mainly treated with immunosuppressants such as cyclophosphamide and corticosteroids, the goal of which is to keep symptoms under control.

One manifestation of SLE is abnormalities in apoptosis, a type of programmed cell death in which aging or damaged cells are neatly disposed of as a part of normal growth or functioning. In SLE, the resolution of the apoptosis is impaired, resulting in cellular debris remaining. During an immune reaction to a foreign stimulus, such as bacteria, virus, or allergen, immune cells that would normally be deactivated due to their affinity for self-tissues can be abnormally activated by signaling sequences of antigen-presenting cells. Thus triggers may include viruses, bacteria, allergens (IgE and other hypersensitivity), and can be aggravated by environmental stimulants such as ultraviolet light and certain drug reactions. These stimuli begin a reaction that leads to destruction of other cells in the body and exposure of their DNA, histones, and other proteins, particularly parts of the cell nucleus. The body's sensitized B-lymphocyte cells will now produce antibodies against these nuclear-related proteins. These antibodies clump into antibody-protein complexes which stick to surfaces and damage blood vessels in critical areas of the body, such as the glomeruli of the kidney; these antibody attacks are the cause of SLE. SLE is a chronic inflammatory disease believed to be a type III hypersensitivity response with potential type II involvement. Impaired clearance of dying cells is a potential pathway for the development of this systemic autoimmune disease. This includes deficient phagocytic activity and scant serum components in addition to increased apoptosis. The clearance of early apoptotic cells is an important function in multicellular organisms. It leads to a progression of the apoptosis process and finally to secondary necrosis of the cells if this ability is disturbed. Necrotic cells release nuclear fragments as potential autoantigens, as well as internal danger signals, inducing maturation of dendritic cells (DCs), since they have lost their membranes' integrity. Increased appearance of apoptotic cells also stimulates inefficient clearance. That leads to maturation of DCs and also to the presentation of intracellular antigens of late apoptotic or secondary necrotic cells, via MHC molecules. Autoimmunity possibly results by the extended exposure to nuclear and intracellular autoantigens derived from late apoptotic and secondary necrotic cells. B and T cell tolerance for apoptotic cells is abrogated, and the lymphocytes get activated by these autoantigens; inflammation and the production of autoantibodies by plasma cells is initiated. A clearance deficiency in the skin for apoptotic cells has also been observed in people with cutaneous lupus erythematosus (CLE).

Oxidative damage mediated by reactive oxygen species results in the generation of deleterious by-products. The oxidation process itself and the proteins modified by these molecules are important mediators of cell toxicity and disease pathogenesis. Aldehydic products, mainly the 4-hydroxy-2-alkenals, form adducts with proteins and make them highly immunogenic. Proteins modified in this manner have been shown to induce pathogenic antibodies in a variety of diseases including systemic lupus erythematosus (SLE), alcoholic liver disease, diabetes mellitus (DM) and rheumatoid arthritis (RA). 8-oxodeoxyguanine (oxidatively modified DNA) and low density lipoproteins (LDL) occur in SLE, a disease in which premature atherosclerosis is a serious problem. Oxidatively modified glutamic acid decarboxylase is important in type 1 DM, while autoantibodies against oxidized LDL are prevalent in Behcet's disease. See, Kurien, B. T. Scofield, R. H., “Autoimmunity and oxidatively modified autoantigens”, Autoimmun Rev. 2008 July; 7(7): 567-573. Published online 2008 May 27. doi: 10.1016/j.autrev.2008.04.019.

Reactive oxygen species (ROS) are oxygen-based molecules possessing high chemical reactivity. These include free radicals (superoxide and hydroxyl radicals) and non-radical species (hydrogen peroxide) which can be produced even at basal conditions by a number of ways. Free radicals are active species containing atoms or molecules with one or more unpaired electrons occupying an outer orbital. They can arise either by the univalent pathway of oxygen reduction or as a consequence of enzymic/non-enzymic reactions. The superoxide anion radical O₂— is formed as a consequence of the one electron reduction of O₂. The two electron reduction product of O₂ in the fully protonated form is hydrogen peroxide (H₂O₂) while the three electron reduction product of O₂ is the hydroxyl radical (OH.). A number of enzymic and non-enzymic reactions reduce oxygen to the more reactive superoxide radical. Superoxide is also released consequent to the in vitro oxidation of a number of compounds. H₂O₂ may be formed consequent to either the divalent reduction of oxygen by the enzymes urate-, D amino acid- and glycolate oxidases or by the univalent reduction of oxygen to superoxide and subsequent conversion of superoxide to hydrogen peroxide by superoxide dismutase. Though hydrogen peroxide is not a free radical by itself, it can lead to the formation of the more dangerous hydroxyl radical via the Fenton type reaction.

Enzymatic (superoxide dismutase (SOD), catalase and the peroxidases) and non-enzymatic (ascorbic acid, reduced glutathione and vitamin E) antioxidant defense systems control ROS production by scavenging or decreasing ROS levels, thereby maintaining an appropriate cellular redox balance. Alterations of this normal balance resulting from elevated ROS production and/or decreased anti-oxidant levels leads to a state of oxidative stress and thus an enhanced susceptibility of membranes and biological molecules to react with free radicals. SOD converts superoxide into H₂O₂ (which is further converted into water by catalase/glutathione peroxidase). Four types of SOD have been identified based on their tissue distribution. SOD1 (copper/zinc containing SOD) is found in the cytoplasm of virtually all eukaryotic cells. SOD2 (manganese containing SOD) is located in the matrix of the mitochondria of all aerobes. Ferrous SOD is mainly located in the cytosol of prokaryotes. SOD3 (extracellular Cu—Zn SOD) is present in mammals in extracellular fluids or is membrane associated. Except for Photobacterium leiognathi and Caulobacter crescentus, prokaryotes do not contain this enzyme.

Stress or any other factor that compromises the activity of antioxidant enzymes may trigger a potentially dangerous pathway of peroxidative damage. Peroxidative damage brought about by free radicals has been shown to be involved in the pathogenesis of several diseases. Increased oxidant stress has been associated with the observed increase in lipid peroxidation in these diseases. Lipid peroxidation has been defined as oxidative degeneration of polyunsaturated fatty acids, set into motion by free radicals.

Oxidation of any polyunsaturated fatty acid is a chain reaction process and can be divided into three stages: initiation, propagation and termination. In the initiation phase a primary reactive radical, abstracts a hydrogen atom from a methylene group of a polyunsaturated fatty acid to start the peroxidation. This leaves an unpaired electron on the carbon, resulting in the formation of a conjugated diene. The carbon-centered fatty acid radicals combine with molecular oxygen, in the propagation phase, yielding highly reactive peroxyl radicals that react with another lipid molecule to form hydroperoxides. Peroxyl radicals are capable of producing new fatty acid radicals, resulting in a radical chain reaction. In this reaction, the peroxyl radicals themselves are converted to stable termination phase products, lipid hydroperoxides. The lipid peroxidation process can result in a number of deleterious end products.

Lipid peroxidation occurs as a consequence of increased oxidative stress resulting from the disruption of the pro-oxidant/antioxidant balance and is an important pathogenic process in oxygen toxicity. The effect is seen indirectly by the decrease in the levels of antioxidant enzymes or antioxidants like ascorbic acid, reduced glutathione or vitamin E. The process of lipid peroxidation releases aldehydic products of lipid peroxidation (α, β-unsaturated aldehydes), mainly the 4-hydroxy-2-alkenals, that can form adducts with free amino groups of lysine and other amino acids. Aldehyde-modified proteins are highly immunogenic.

Several human diseases are autoimmune in nature resulting from the abrogation of self-tolerance. Autoimmune disease may be either organ-specific or tissue specific. Organ specific diseases include type 1 diabetes, thyroiditis, myasthenia gravis, primary biliary cirrhosis and Goodpasture's syndrome while systemic diseases include rheumatoid arthritis, progressive systemic sclerosis and systemic lupus erythematosus. Nearly all these diseases are characterized by the presence of autoantibodies. Autoantibodies have been shown to be typically present several years prior to diagnosis of SLE and type I diabetes and serve as markers for future disease. Inflammation, infection, drugs, ROS, environmental factors induce formation of neo-antigens. Oxidative damage has been implicated in several autoimmune diseases, including systemic lupus erythematosus. Although there may be no active tolerance to many intracellular self-antigens, immune tolerance to self is maintained by elimination of self-reactive lymphocytes in the thymus during the development of the immune system and by rendering the T lymphocytes that bind self-antigens anergic in the periphery. The disruption of self-tolerance, which results in the appearance of autoreactive lymphocytes, results in autoimmunity. This autoimmune response is generally divided into three kinds, namely B-cell dominant, T-cell dominant, and combinational types. Autoimmune hemolytic anemia and myasthenia gravis belong to the category of B-cell dominant autoimmune diseases while experimental autoimmune encephalomyelitis, insulin-dependent diabetes mellitus and the collagen-induced arthritis are T-cell dominant autoimmune diseases. SLE arises from the emergence of both autoreactive T and B cells with an etiology. Once immune tolerance to one component is abrogated, B- and T-cell responses can diversify to other components of the macromolecule with the recognition of other epitopes in the intact particle.

Free radical or ROS mediated damage occurs in SLE and other diseases. Significantly higher 4-hydroxy-2-nonenal-modified protein levels occur in children with lupus. SOD1 activity was decreased in lupus. Malondialdehyde and conjugated dienes were significantly elevated in lupus patients compared to controls. Antibodies to SOD1 were significantly increased in SLE patients and are potentially responsible for the increased oxidative damage seen. Oxidatively modified LDL's have been shown to elicit autoantibodies and oxidant stress has been attributed to the development of anti-phospholipid antibodies. Elevated levels of anti-oxLDL autoantibodies occur in SLE patients and studies show that anti-oxLDL positively correlate with antiphospholipid antibodies and anti-β-2-glycoprotein. Antibodies to oxLDL that are cross-reactive with phosopholipids are thought to be due to binding to oxidized phospholipids. Circulating oxLDL/β-2-glycoprotein complexes and IgG immune complexes containing oxLDL/β-2-glycoprotein occur in SLE and/or phospholipid syndrome. Increased levels of 8-oxo-deoxyguanine (8-oxodG) have been found in lymphocytes from patients with SLE. An investigation of blood monocytes from patients with SLE showed an impairment in the removal of 8-oxodG as a result of a deficient repair system.

Rheumatoid arthritis (RA) is an autoimmune disorder characterized by synovitis, chronic inflammation of the joints, erosion of the cartilage and bone. The exact pathogenesis in still unknown and treatment is non-curative. The presence of shared epitope QKRAA on the HLA-DRβ chain and the presence of rheumatoid factor (RF) have served as long-term outcome predictors of RA.

Type 1 diabetes mellitus is an autoimmune disease that is organ-specific with T cell mediated destruction of β cells of the pancreatic islet cell and ROS involvement. Studies have demonstrated that protein glycation, oxidation and nitration are elevated in cellular and extracellular proteins in diabetes. Glycation of proteins, oxidation of proteins and nitration is thought to contribute to vascular cell dysfunction and the development of retinopathy, nephropathy and neuropathy (microvascular diabetic complications). The quality and functional integrity of proteins are maintained by the cellular machinery by the degradation and replacement of damaged proteins (oxidation and glycation are the main types of physiological damage). The glycated, oxidized and nitrated amino acid residues are liberated by cellular proteolysis as free adducts and released into plasma for excretion into the urine. Thus, the changes in plasma concentrations and excretion of glycation, oxidation and nitration adducts may reflect damage to tissues in diabetes, yielding new markers of the damaging effects of hyperglycemia. In a study of 21 type 1 diabetes mellitus patients and 12 control subjects, the concentrations of protein glycation, oxidation and nitration adduct residues were found to be increased in type 1 diabetes mellitus patients compared to normal controls (up to 3-fold in plasma protein and up to 1-fold in hemoglobin; except for decrease in pentosidine and 3-nitrotyrosine residues in hemoglobin). However, the same study found that the concentrations of protein glycation and oxidation free adducts increased up to 10-fold in plasma while urinary excretion was found to increase up to 15-fold in diabetic patients. Type 1 diabetes mellitus is also distinguished by the presence of a number of autoantigens. Glutamic acid decarboxylase is one of the major, and most well characterized autoantigens. Treatment of p cell lysates with copper sulphate and iron sulphate produces high molecular weight complexes of glutamic acid decarboxylase independent of disulphide double bonds. Sera from patients with type 1 diabetes mellitus bind these complexes much more strongly than they bind the glutamic acid decarboxylase monomer. Thus, oxidative modification of glutamic acid decarboxylase may be important in type 1 diabetes mellitus patients pathogenesis.

Scleroderma or systemic sclerosis is a systemic autoimmune disease that affects several organs including skin, lung and kidneys leading to widespread tissue fibrosis as well as vasculopathy. Patients affected with systemic sclerosis have autoantibodies that bind several autoantigens. Addition of ferrous sulphate to HeLa cell extracts fragment specific scleroderma autoantigens in a unique way. RNA polymerase II, topoisomerasel, upstream binding factor and the 70 kD protein of U1 RNA are fragmented in this manner and this fragmentation was inhibited by metal ion chelators. Some of these fragments were also generated by copper mediated oxidation. The authors also investigated intact keratinocytes exposed to supra-physiological concentrations of copper in which oxidation was started by hydrogen peroxide addition. Topoisomerase was shown this way to be cleaved into the 95 kD fragment that was previously observed with in vitro studies. The authors propose that perfusion-reperfusion injury found in scleroderma in the presence of metal ions may produce these oxidatively modified autoantigens. Such modified antigens might initiate the autoimmune process through cryptic epitopes. Such a scenario, however, assumes the fact that autoantibodies arise consequent to the pathologic process. It is now well established that autoantibodies precede disease manifestations in many autoimmune diseases. This systemic disease is characterized by the presence of ocular occlusive vasculitis and thrombosis, and anterior or posterior uveitis in conjunction with oral aphthae, genital ulceration and cutaneous lesions. Excessive production of ROS is present in Behcet's disease, with associated significant increase in malondialdehyde production and decreased glutathione peroxidase activity. Another study showed significantly elevated levels of autoantibodies against oxidized LDL and lipid hydroperoxides in a group of patients with Behcet's disease compared to healthy controls. In addition this study found that erythrocyte SOD, catalase and plasma glutathione peroxidase activities were significantly lower in Behcet's disease patients compared to controls. The decrease in these antioxidant enzymes would be responsible for the increased oxidative stress occurring in Behcet's disease, the susceptibility of LDL to oxidation and thus may predispose these patients to atherothrombotic events.

The role of free radicals in the pathogenesis and development of diseases is well documented. Generation of ROS and enzymatic and non-enzymatic control of these harmful molecules is an ongoing process. Antibodies to antioxidant enzymes could result in the disruption in this balance resulting in oxidative stress, which is turn leads to pathological changes. This could lead to oxidatively modified autoantigens that serve as neo-antigens in promoting loss of tolerance to self. Immunization with modified autoantigens has shown accelerated epitope spreading and induction of disease. Kurien et al state, “Administration of antioxidants or other dietary modulations is not studied in autoimmune disease, but could be helpful in preventing or ameliorating disease although results in cardiovascular disease are disappointing”. Gutteridge J M C, Westermarck T, Halliwell B. Oxygen radical damage in biological systems. In: Johnson J E, Walford R, Harman D, Miquel J, editors. Free Radicals, Aging, and Degenerative Diseases. New York: Alan R. Liss; 1985. p. 99 (see cited references).

Systemic lupus erythematosus (SLE) is characterized by imbalance redox state and increased apoptosis. The activation, proliferation and cell death of lymphocytes are dependent on intracellular levels of glutathione and controlled production of reactive oxygen species (ROS). See, Dilip Shah, Sangita Sah, and Swapan K. Nath. Interaction between glutathione and Apoptosis in Systemic Lupus Erythematosus. Autoimmun Rev. 2013 May; 12(7): 741-751. Published online 2012 Dec. 29. doi: 10.1016/j.autrev.2012.12.007. Changes in the intracellular redox environment of cells, through oxygen-derived free radical production known as oxidative stress, have been reported to be critical for cellular immune dysfunction, activation of apoptotic enzymes and apoptosis. The shift in the cellular GSH-to-GSSG redox balance in favor of the oxidized species, GSSG, constitutes an important signal that can decide the fate of the abnormal apoptosis in the disease.

A key issue in the pathogenesis of lupus is how intracellular antigens become exposed and targeted by the immune system. In this regard, excessive production of ROS, altered redox state and a defect in regulation of apoptosis are considered as factors involved in the production, expansion of antibody flares and various clinical features in SLE. The depletion of intracellular glutathione is an indicator for ROS formation and may be involved in dysregulation of apoptosis in disease. The oxidative damage mediated by ROS resulting in the defect in control of apoptosis or programmed cell death and delayed clearance of apoptotic cells may prolong interaction between ROS and apoptotic cell macromolecules generating neoepitopes that subsequently broad spectrum of autoantibody formation leading to the tissue damage in SLE. An increase in MDA-modified proteins, anti-SOD and anti-catalase antibodies in the sera of SLE patients support a critical role for oxidative stress in disease development. The positive relationships between oxidative stress markers and apoptosis reinforce the contribution of oxidative stress in the perturbation of apoptosis in SLE.

A diverse number of stimuli have been shown to induce apoptosis, many of which are also known to compromise the fine balance between intracellular oxidants and their defense systems. Oxidative stress is believed to play a major role in the initiation and progression of autoimmune disease by excessive free radical formation. An increase in ROS production or a decrease in ROS-scavenging capacity due to exogenous stimuli or endogenous metabolic alterations can disrupt redox homeostasis, lead to an overall increase intracellular ROS levels, or oxidative stress. Among the ROS, —OH is the most potent damaging radical, and can react with all biological macromolecules (lipids, proteins, nucleic acids and carbohydrates). It can lead to the formation of DNA-protein cross-links, single- and double-strand breaks, base damage, lipid peroxidation and protein fragmentation. This oxygen species may penetrate cellular membranes and react with nuclear DNA. Murine models of SLE demonstrate abnormally high levels of NO compared with normal mice, whereas systemic blockade of NO production reduces disease activity. Elevated serum nitrate levels correlate with indices of disease activity and, along with serum titers of anti-(ds DNA) antibodies, serve as indicators of SLE. Excessive oxidative stress is thought to play an important role in the pathogenesis of autoimmune diseases by enhancing inflammation, inducing apoptotic cell death and breaking down immunological tolerance. Free radical production and altered redox status can modulate expression of a variety of immune and inflammatory molecules leading to inflammatory processes, exacerbating inflammation and affecting tissue damage. ROS generation also provides oxidant for thiol oxidation or peroxynitrite formation which can be a basis for antibody modification. Convincing evidence for the association of oxidative/nitrosative stress and SLE diseases has been shown by increased levels of validated biomarkers of oxidative stress in the disease. Increased levels of 8-oxodG, a marker of oxidative DNA damage in the immune complex derived DNA, have been found in lymphocytes and serum from SLE patients, reinforcing ROS in disease etiology. The level of protein oxidation markers correlating with severity of disease in SLE patients further supports the role of protein oxidation in SLE. Elevated levels of F2 isoprostanes (prostaglandin-like substances derived from lipid peroxidation) in serum and urine from SLE patients have been reported. It has been reported that OH, could lead to neoantigens like OH damaged human serum albumin (HSA), which in turn could initiate autoimmunity in SLE. These reports support the role of oxidative stress in the pathogenesis of SLE.

The primary target of ROS is lipids in the cell membrane and lipid peroxidation (LPO) impairs cell structure and function. An increase in malondialdehyde (MDA), a product of lipid peroxidation, has been reported in serum/plasma/erythrocyte as well as in lymphocytes in patients with SLE. The increased level of lipid peroxidation was positively correlated with severity of the disease and organ damage especially in nephritis patients. All cell types, including lymphocytes and other immune cells, have a complex machinery of antioxidant enzymes (superoxide dismutase, catalase, glutathione peroxidase, glutathione reductase, thioredoxin etc.) and antioxidant molecule (reduced glutathione, vitamins) for regulating oxidant reactions in the cells prevent free radical mediated cytotoxicity. The circulating human erythrocytes are able to scavenge O₂.— and H₂O₂ by SOD, CAT, and GPx-dependent mechanisms may be important in regulating such reactions. The first line of defense against ROS is provided by SOD which catalyzes dismutation of O₂.— into H₂O₂. The H₂O₂ is then transformed into H₂O and O₂ by catalase. GPx is a selenoprotein that reduces lipidic or nonlipidic hydroperoxides as well as H2O2 utilizing glutathione. The activity of glutathione peroxidase is controversial in SLE patients however, and most showed decreased activity of GPx in SLE patients.

Adequate concentrations of glutathione are required for a variety of functions, including protection of the cell from oxidative damage quenching of oxidant species, lymphocyte activation, natural killer cell activation and lymphocyte-mediated cytotoxicity. The depletion of intracellular glutathione has been associated with many autoimmune inflammatory diseases including SLE. A decrease in the level of intracellular GSH showed a correlation with the severity of disease especially with nephritis patients. Decreased intracellular GSH may be ascribed to ROS-induced GSH oxidation or GSH export from cells.

The effect of ROS is limited by the presence of various regulatory systems that maintain redox homeostasis. A relatively large number of compounds have been shown to possess some measurement of antioxidant activities. They maintain a balance between the production and metabolism of ROS and protect the cell from oxidative damage. The antioxidant enzymes include SOD, CAT and glutathione related enzymes; GPx, GR and GST. The non-enzymatic scavengers are vitamins E, C and A and thiol containing compounds such as glutathione. Reduced glutathione (L-γ-glutamyl-L-cysteinylglycine) is the most prevalent cellular thiol and the most abundant low molecular weight peptide present in all cells. The role of GSH as a reductant is extremely important in the highly oxidizing environment of the erythrocyte. GSH levels in human tissues normally range from 0.1 to 10 mM, most concentrated in the liver (up to 10 mM), spleen, kidney, lens, erythrocytes and leucocytes. In healthy cells and tissues, more than 90% of the total glutathione pool is in the reduced form (GSH) and less than 10% exists in the oxidized form (GSSG). Glutathione is required for many critical cellular processes and plays a particularly important role in the maintenance and regulation of the thiol-redox status of the cell. The GSH/GSSG ratio is a useful measurement for determining oxidative stress and changes in this ratio appear to correlate with cell proliferation, differentiation and apoptosis. This led to attention to the role of thiol status in the onset and progression of autoimmune and inflammatory diseases, including rheumatoid arthritis and, SLE as well as the effectiveness of thiol repletion therapies in the treatment of these diseases. Cellular GSH levels affect T helper cell maturation, T cell proliferation, as well as susceptibility to ROS secreted by inflammatory cells. Additionally, many correlations exist between immune system dysfunction and alterations in GSH levels in the cells. It is reported that GSH depletion in antigen presenting cells inhibits Th1-related cytokine production like IFN-γ and IL-12 and supports the Th2-mediated humoral immune response. Furthermore, when antigen presenting cells have high intracellular GSH levels they secrete cytokines that favor the development of Th1 cells. In addition, it is reported that specific cytokines can alter GSH levels in antigen presenting cells. Exposure to IFN-γ, a Th1 cytokine, resulted in increased GSH levels, whereas exposure to IL-4, a Th2 cytokine, resulted in decreased intracellular GSH. Because GSH has a significant impact on the immune system's ability to activate the appropriate Th response, altering its levels may have significant implications in Th1/Th2-related diseases like SLE. Glutathione peroxidase is a tetrameric protein (85 KDa), which has four atoms of selenium bound as selenocysteine moieties that confer catalytic activity. It has a lower Kₘ value for H₂O₂ than CAT and considered more important when low amounts of H₂O₂ are generated. It plays an important role in the defense mechanism against oxidative damage in erythrocytes by catalyzing the reduction of H₂O₂ and variety of lipid hydro-peroxides using GSH as the reducing substrate. In SLE patients, decreased activity of GPx leads to a change in redox ratio in favor of oxidized glutathione. Glutathione, a strong natural antioxidant molecule not only controls oxidative stress of the cells but is also involved in regulation of apoptosis pathway and cytokine network in SLE.

Apoptosis is a form of actively induced programmed cell death, with the characteristic features of chromatin condensation, DNA fragmentation and apoptotic body formation. Apoptotic bodies composed of numerous nucleolus bodies and organelles are normally removed by phagocytes as soon as they are formed. Failure to remove apoptotic bodies leads to the release of autoantigens that may cause autoimmunity. Progressive studies on SLE demonstrated that lymphocyte apoptosis might play an important role in the pathogenesis of disease. During the process of apoptosis, release of excessive quantity of intact nucleosomes has been suggested to be a source of nuclear antigens that drive an immune response, inducing anti-DNA and anti-histone antibody production. If the apoptotic cells are not phagocytosed immediately, they undergo post-translational modification altering antigenicity that may provide a source of nuclear antigens to drive the autoantibody response in SLE. Tolerance of self-antigens requires the deletion of autoreactive T- and B-cells by apoptosis. Therefore defects in inducing apoptosis could lead to the persistence of autoreactive T- or B-cells. Thus, defective apoptosis leading to prolonged survival of pathogenic lymphocytes could be one cause of SLE. A recent study on SLE patients showed increased levels of Fas/FasL in SLE patients related to depletion of intracellular glutathione. Taken together, apoptosis of lymphocytes may be defective in patients with SLE and Fas/FasL-mediated signaling pathways could be crucial in the process. Altered lymphocyte apoptosis in patients with SLE could contribute to an overload of nucleosome in circulation that could initiate an autoimmune response that might break tolerance, resulting in the autoimmune phenomena.

A decrease in cellular GSH concentration has long been reported to be an early event in the apoptotic cascade induced by death receptor activation, mitochondrial apoptotic signaling, and oxidative stress. Convincing evidence showed that GSH depletion during apoptosis is an indicator for ROS formation and oxidative stress and may be tied to pathogenesis in many autoimmune diseases including SLE. Changes in the intracellular thiol-disulfide (GSH/GSSG) balance are considered major determinants in the redox status/signaling of the cell. GSH constitutes the major intracellular antioxidant defense against RS and oxidative stress. GSH has been shown to scavenge a wide variety of RS, including superoxide anion (O₂.—), hydroxyl radical (—OH), singlet oxygen (¹O₂), protein-, and DNA radicals, by donating electrons and becoming oxidized to glutathiyl radical (GS.). Generation of disulfide bonds between two GSH leads to further formation of GSSG. GSH also catalytically detoxifies cells from peroxides such as hydroperoxides (H₂O₂), peroxynitrite (OONO—), and lipid peroxides (LOO.) by the action of GSH peroxidases (GPX) and peroxiredoxins (PXR). Accumulation of GSSG on oxidative stress has been observed to be toxic to the cell. GSSG has been shown to directly induce apoptosis by the activation SAPK/MAPK pathway. GSH depletion in response to oxidants has been widely reported, and linked to cell death. GSH is essential for cell survival as demonstrated by observations that glutamate cysteine ligase (GCL) knockout mice die from massive apoptotic cell death, and that the knockdown of GCL in distinct cell types induces time-dependent apoptosis. GSH levels have been shown to influence caspase activity, transcription factor activation, Bcl-2 expression and function, ceramide production, thiol-redox signaling, and phosphatidylserine externalization. A remarkable feature of cells undergoing apoptosis is that they rapidly and selectively release a large fraction of their intracellular GSH into the extracellular space. GSH peroxidase (GPX) has been shown to protect against apoptosis induced by Fas activation. The apoptosis-inducing effects can be blocked by glutathione and N-acetylcysteine. Glutathione depletion has been reported to involve in extrinsic/death receptor as well as intrinsic pathway of apoptosis.

Induction of apoptosis via the extrinsic pathway is triggered by the activation of the death receptors Fas (CD95/Apo-1), TNF-related apoptosis-inducing ligand (TRAIL) receptors 1 and 2 (DR4/DR5), and TNF receptor 1 (TNFR1) by their respective ligands FasL, TRAIL, and TNF-α. Activation of death receptors leads to formation of the death-inducing signaling complex, which includes the Fas-associated death domain (FADD), initiator caspase 8 or 10, and the cellular FADD-like interleukin-1 beta-converting enzyme (FLICE)-inhibitory protein (FLIP) leading to the activation of initiator caspases. Activation of NFkB antagonizes programmed cell death induced by TNFR1, and GSH depletion has been shown to down-regulate TNF-induced NFkB activation and sensitize to apoptotic cell death. GSH depletion is necessary for the formation of the apoptosome and also triggers cell death by modulation of the permeability transition pore of the mitochondria and the activation of executioner caspases. In addition, GSH depletion activates the intrinsic apoptotic pathway initiator Bax and Cyt C release. Released Cyt C requires cytosolic GSH levels to be depleted for its pro-apoptotic action. Depletion of intracellular GSH also overcomes Bcl-2-mediated resistance to apoptosis. The antiapoptotic role of Bcl-2 has been linked to GSH content by several studies, where it was reported that Bcl-2 regulates GSH content and distribution in different cellular compartments. A recent study suggests that Bcl-2 regulates mitochondrial GSH content by a direct interaction of the BH3 groove with GSH, while the antiapoptotic effect of Bcl-xl has also been attributed to the regulation of GSH homeostasis by preventing GSH loss. In SLE patients, depletion of glutathione has been associated with various immune abnormalities including deregulation of apoptosis, abnormal cytokine and chemokine production and various clinical features. There are several lines of evidence correlating the depletion of intracellular glutathione with generation of ROS/RNS and progression of apoptosis in SLE patients. It has been reported that glutathione levels were diminished in RBC and total lymphocyte as well as lymphocyte subsets in SLE patients. Depletion of glutathione is correlated with severity of the disease and allied with oxidative stress and apoptosis. The diminished levels of glutathione in the RBC and lymphocytes positively associated with increased levels of oxidative stress makers such as ROS, lipid peroxidation in SLE patients. A negative association of the levels of GSH levels with apoptosis of T lymphocytes, CD4+, CD8+T lymphocyte sub-sets and intracellular activated caspase-3 may support the role of reduced glutathione in the alteration of T lymphocyte apoptosis in the disease state. These results suggest that glutathione played a role in depletion of CD4+T lymphocyte in SLE patients. The role of glutathione as a therapeutic molecule to replenish depleted glutathione has been related to reduction in autoantibody. It has been show diminished GSH/GSSG ratios in the kidneys of 8-month-old versus 4-month-old (NZB×NZW) F1 mice, and treatment with N-acetylcysteine (NAC), a precursor of GSH and stimulator of its de novo biosynthesis, prevented decline of GSH/GSSG ratios, reduced autoantibody production and development of glomerulonephritis and prolonged survival of (NZB×NZW) F1 mice. Intracellular glutathione has been shown to be involved in regulating several immune mechanisms in human body. While GSH scavenges .OH, ¹O₂, and NO directly, it catalytically detoxifies hydrogen peroxides (H₂O₂), OONO—, and lipid peroxides by activation of glutathione peroxidases. Perricone and his group have shown that modulation of intracellular glutathione can inhibit complement-mediated damage in autoimmune diseases. Because glutathione is the major intracellular antioxidant defense within a cell, it is proposed that its depletion might be a prerequisite for modulating the apoptotic machinery in autoimmune disease like SLE. Inhibition of GSH depletion by either high extracellular GSH or NAC may prevent increased ROS formation and control abnormal apoptosis as well as several other abnormal immune responses, cytokine as well as chemokine production in SLE patients.

Thioredoxin

The thioredoxin (Trx) system and the GSH-glutaredoxin (Grx) system are two major thiol dependent disulfide reductases in the cells, which transfer the electrons from NADPH to their substrates. The two thiol dependent electron transferring pathways play critical roles in defense against oxidative stress by reducing methionine sulfoxide reductases (MSR) to repair proteins or peroxiredoxins (Prx) to remove peroxides. They are also electron donors for ribonucleotide reductase (RNR), which is essential for the production of deoxyribonucleotides and DNA. The thioredoxin (Trx), thioredoxin reductase (TrxR), and NADPH are together called the thioredoxin system, which serves as a hydrogen donor for ribonucleotide reductase and has a general powerful disulfide reductase activity. The thioredoxin system is present in cells and in all forms of life. Thioredoxin reductase (TrxR) is a dimeric FAD containing enzyme that catalyzes the reduction of its main protein substrate oxidized thioredoxin, to reduced thioredoxin at the expense of NADPH. The enzyme mechanism involves the transfer of reducing equivalents of NADPH to a redox active site disulfide via an FAD domain. Thioredoxin reductase from Escherichia coli with subunits of 35 kDa has been extensively characterized. X-ray crystal structure reveals that the active site disulfide is located in a buried position in the NADPH domain and suggests that it should undergo a large conformational change to create a binding site for Trx-S₂ and reduction by a dithiol-disulfide exchange. Trx system is composed with thioredoxin reductase (TrxR), Trx and NADPH. Trx is ubiquitous in all living organisms with its conserved CGPC active site and the Trx fold (1). In contrast, the TrxRs in mammalian cells and bacteria showed notable differences in structure and reaction mechanism. Bacteria have a smaller (70 kDa) sulfur-dependent enzyme whereas human and animal cells have a large (115 kDa) selenocysteine-containing enzyme. Moreover, many pathogenic bacteria contain distinct thiol-dependent redox systems. Particularly, some pathogenic bacteria lack glutathione (GSH) and glutaredoxin (Grx) and thus TrxR and Trx are essential for DNA synthesis and the Trx system should be a suitable target for development of antibacterial drugs. Thioredoxin reductase is a ubiquitous enzyme present in all cells. However, the enzyme is often over-expressed in tumor cells compared to normal tissues, and tumor proliferation seems to be crucially dependent on an active thioredoxin system, making it a potential target for anticancer drugs. Over the last decade a number small organic and organometallic molecules that include platinum and gold containing complexes naphthoquinone spiroketal based natural products, different naphthazarin derivatives, certain nitrosoureas and general thiol (or selenol) alkylating agents such as 4-vinylpyridine, iodoacetamide, or iodoacetic acid have been identified as inhibitors of Trx or TrxR or both. Engman et al. have reported the inhibition of mammalian thioredoxin reductase by diaryldichalcogenides and organotellurium compounds. However, no inhibition has been presented for bacterial TrxR. Thioredoxins together with glutaredoxins are the two dithiol hydrogen donors for the essential enzyme ribonucleotide reductase required for DNA synthesis. The two enzymes glutathione reductase (GR encoded by the gor gene) and thioredoxin reductase (TrxR encoded by the trxB gene) in E. coli are central in electron transport from NADPH. Thioredoxin reductase from human and animal cells is a large selenoenzyme and very different from the enzymes present in all prokaryotes. In contrast to the mammalian enzymes the E. coli enzyme is highly specific and utilizes a different mechanism with an involvement of protein conformation change as mentioned above. Thioredoxin reductase (TrxR), catalyzes the electron donation from NADPH via thioredoxin (Trx) to ribonucleotide reductase (RNR) and may be essential for DNA synthesis if no other system is present. Cytosolic Trx is a highly conserved 12 kDa protein whereas the cytosolic TrxRs from mammalian and bacterial, e.g. Escherichia coli, are very different in their structure and catalytic mechanisms, with mammalian TrxR being a large selenoenzyme.

Isoselenazol or Isothiazol Derivatives

Ebselen, 2-phenyl-1,2-benzoisoselenazol-3(2H)-one is an antioxidant and anti-inflammatory selenoorganic compound used in clinical trials against e.g. stroke. It is thus known to be safely administered to humans. Ebselen and ebselen diselenide have been reported as substrates for mammalian thioredoxin reductase (3a) and its reaction mechanisms have been published. There are several reports of synthesis of substituted benzisoselenazol-3(2H)-ones. Some of these compounds were reported as inhibitors of viral cytopathogenicity and active immunostimulants inducing cytokines, such as interferons (IFNs), tumor necrosis factors (TNFs) and interleukin (IL-2) in human peripheral blood leukocytes. However, none of the reports indicates thioredoxin reductase activity.

It has been shown that ebselen, which has been known as a glutathione peroxidase (GSPx) mimic (1), is a substrate for human and mammalian thioredoxin reductase and a highly efficient oxidant of reduced thioredoxin. This strongly suggested that the thioredoxin system (NADPH, thioredoxin reductase and thioredoxin) is the primary target of ebselen, since a highly efficient reduction of hydroperoxides was given by ebselen in the presence of the thioredoxin system.

The cyclic-di-GMP (cdiGMP) signaling pathway regulates biofilm formation, motility, and pathogenesis. Pseudomonas aeruginosa is an important opportunistic pathogen that utilizes cdiGMP-regulated polysaccharides, including alginate and pellicle polysaccharide (PEL), to mediate virulence and antibiotic resistance. CdiGMP activates PEL and alginate biosynthesis by binding to specific receptors including PelD and Alg44. Ebselen was identified as an inhibitor of cdiGMP binding to receptors containing an RxxD domain including PelD and diguanylate cyclases (DGC). Ebselen reduces diguanylate cyclase activity by covalently modifying cysteine residues. Ebselen oxide, the selenone analogue of ebselen, also inhibits cdiGMP binding through the same covalent mechanism. Ebselen and ebselen oxide inhibit cdiGMP regulation of biofilm formation and flagella-mediated motility in P. aeruginosa through inhibition of diguanylate cyclases. Lieberman, O. J. et al. “High-Throughput Screening Using the Differential Radial Capillary Action of Ligand Assay Identifies Ebselen As an Inhibitor of Diguanylate Cyclases”, ACS Biology 2014, 9, 183-192.

It was previously discovered that that ebselen [2-phenyl-1,2 benzisoselenazol-3(2H)-one], (EbSe) which is a substrate of mammalian TrxR and an competitive reversible inhibitor of bacterial TrxR, displays selective antibacterial activity toward certain bacteria lacking glutathione. The pathogenic bacteria including Helicobacter pylori, Mycobacterium tuberculosis, and Staphyloccus aureus exhibit high sensitivity to ebselen. The thioredoxin (Trx), thioredoxin reductase (TrxR), and NADPH are together called the thioredoxin system, which serves as a hydrogen donor for ribonucleotide reductase and has the most general powerful disulfide reductase activity. The thioredoxin system is present in cells and in all living systems. Thioredoxin reductase (TrxR) is a dimeric FAD containing enzyme that catalyzes the reduction of its main protein substrate oxidized thioredoxin to reduced thioredoxin, at the expense of NADPH. The enzyme mechanism involves the transfer of reducing equivalents of NADPH to a redox active site disulfide via FAD domain. Thioredoxin reductase from Escherichia coli with subunits of 35 kDa has been extensively characterized. X-ray crystal structure reveals the active site disulfide located in a buried position in the NADPH domain, and suggests that it should undergo a large conformational change to create a binding site for Trx-S₂ and reduction by a dithiol-disulfide exchange.

Thioredoxin reductase is a ubiquitous enzyme present in all living cells. However, the enzyme is often over-expressed in tumor cells compared to normal tissues, and tumor proliferation seems to be crucially dependent on an active thioredoxin system, making it a potential target for anticancer drugs. Over the last decade a number small organic and organometallic molecules that include platinum and gold containing complexes, naphthoquinone spiroketal based natural products, different naphthazarin derivatives, certain nitrosoureas, and general thiol (or selenol) alkylating agents such as 4-vinylpyridine, iodoacetamide, or iodoacetic acid have been identified as inhibitors of Trx or TrxR or both. Engman et al. have reported the inhibition of thioredoxin reductase by diaryldichalcogenides and organotellurium compounds.

Ebselen and ebselen diselenide have been reported as substrates for mammalian thioredoxin reductase and its reaction mechanism. Using glutathione as the reductant, the H₂O₂ reductase activity of ebselen was compared with that in the presence of the mammalian thioredoxin system. Formation of ebselen diselenide may serve as a dose-dependent storage form of ebselen, which can be relatively slowly activated to the catalytically active selenol by the mammalian thioredoxin system. The studies were extended to E. coli TrxR, and surprisingly, ebselen was found to inhibit E. coli TrxR. These findings lead to a search for the new organoselenium compounds containing the basic structure of ebselen, to study their reactivity with thioredoxin reductase. There are several reports of synthesis of substituted benzisoselenazol-3(2H)-ones. Some were reported as inhibitors of viral cytopathogenicity and active immunostimulants inducing cytokines, such as interferons (IFNs), tumor necrosis factors (TNFs) and interleukin (IL-2) in human peripheral blood leukocytes. 2-(4-caroboxyphenyl)benzisoselenazol-3(2H)-one was found to be potent and selective inhibitor of endothelial nitric oxide synthase. However, none of the reports indicates thioredoxin reductase activity.

Ebselen, a small isoselenazol drug well known for its antioxidant and anti-inflammatory properties, also has antibacterial properties. The mechanism has been unknown and there is a remarkable difference in sensitivity between Staphyloccus aureus being a 100-fold more sensitive than E. coli. The growth of methicillin resistant Staphylococcus aureus was shown to be inhibited by 0.20 μg per ml of ebselen, whereas strains of Enterobacteriaceae like E. coli NHHJ were much more resistant requiring up to 50 μg per ml. The MIC for 90% of S. aureus strains was 1.56 μg per ml and the drug was bacteriocidal. Control of bacterial infection using chemotherapeutic principles and antibiotics are based on inhibition of cell wall synthesis, protein synthesis and other metabolic pathways. The presently used drugs have limitations and resistant bacterial infections is an increasing problem as evident by development of vancomycin and methicillin resistant bacteria. Since genetic material in the form of DNA is common to all microorganisms, inhibition of DNA synthesis is an attractive principle. In addition, drugs interrupting the defense of bacteria against oxidative stress should be a useful principle for developing new antibacterial agents. The thioredoxin system, including thioredoxin (Trx), thioredoxin reductase (TrxR) and NADPH, is the most powerful protein disulfide reductase in cells. Together with the glutaredoxin system, including glutaredoxin (Grx), glutathione (GSH), glutathione reductase (GR) and NADPH, thioredoxins are important hydrogen donors of ribonucleotide reductase for DNA synthesis and play key roles in cell redox regulation and growth control.

Thioredoxin reductase is one of those few examples of enzymes where the same reaction is catalyzed by more than one structure and mechanism. Extensive studies on the features and redox properties of TrxR from various organisms resulted in the classification of two TrxRs, one from higher eukaryotes with high molecular weight and structurally resembles the other oxidoreductases; the other from prokaryotes, fungi, and plants with low molecular weight and distinct in structures and catalytic mechanism. Thus the striking difference between the enzymes would make them ultimate targets for novel antibiotic drug designs although this has not yet been reported.

The TrxR from mammalian is a large selenoprotein with homodimer of 55 kD per subunits and a structure closely related to glutathione reductase but with an elongation containing a catalytically active selenol-thiol/selenosulfide in the conserved C-terminal sequence Gly-Cys(496)-Sec(497)-Gly, and thus a wide substrate specificity. The bacterial counterpart of TrxR is however a non-selenoprotein with homodimer of 35 kD per subunits. Each E. coli TrxR monomer consists of an NADPH-binding domain and an FAD binding domain connected by a double-stranded R-sheet. The active site Cys (135)-Ala-Thr-Cys(138) is located in the NADPH domain. A well-recognized characteristic of the E. coli enzyme is its large conformational change during catalysis. In its 3-D structure, the flow of electrons from NADPH to the active-site disulfide via the flavin can only be possible if the NADPH domain graphically rotating over 67° relative to the FAD domain, allowing an efficient hydride transfer from NADPH to FAD (the nicotinamide ring and the isoalloxazine would be in close contact) and simultaneously exposing the redox-active disulphide to the surface of the protein, accessible for the substrate. Mammalian TrxRs are large dimeric selenoproteins (Mᵣ 114.000), with structures closely related to glutathione reductase, but with a C-terminal 16 amino acid elongation containing a unique catalytically active conserved sequence Gly-Cys-Sec-Gly. Mammalian thioredoxin reductases have a remarkably wide substrate specificity. E. coli TrxR is smaller (Mr 70.000/dimer), with the active-site Cys-Ala-Thr-Cys disulfide loop located in the NADPH domain. During catalysis, a large conformational change is required, i.e., from FO (flavin oxidation by disulphide) to FR (flavin reduction by NADPH) form.

Ribonucleotide reductase is a universal enzyme, which for aerobic organisms supply all four deoxyribonucleotides required for DNA synthesis de novo, for either replication or repair. Electrons for the reduction ultimately are from NADPH via either thioredoxin or glutaredoxin. These two small protein thiol electron donors are reduced by separate pathways. Thioredoxin is reduced by thioredoxin reductase, and glutaredoxin by the tripeptide glutathione (GSH), which is present in high millimolar concentrations in most cells. Oxidized glutathione (GSSG) is reduced by glutathione reductase.

Whereas, there are general overall similarities between thioredoxin, glutaredoxin and ribonucleotide reductase in bacteria and human and other mammalian cells, there are fundamental differences between thioredoxin reductase enzymes. Thus, the enzyme is by convergent evolution either low molecular weight specific enzymes like that in E. coli or other bacteria or a high molecular weight selenocysteine-containing enzyme with broad specificity like the three isozymes in human cells.

Ebselen, 2-phenyl-1,2-benzoisoselenazol-3(2H)-one, is an isoselenazol well known for its antioxidant and anti-inflammatory properties and is widely used in laboratories as peroxide reducing antioxidant in in vivo models and has been proved in clinical trials against acute ischemic stroke. We have previously shown that ebselen and its diselenide are substrates for mammalian TrxR and efficient oxidants of reduced Trx forming the ebselen selenol, the active form of ebselen with its hydrogen peroxide reductase activity. The mechanism of antioxidant action of ebselen, together with its diselenide, was mainly through its interactions with the mammalian TrxR and Trx, providing the electrons for the reduction of hydrogen peroxide from NADPH. In the present invention we have discovered that ebselen, however, is not a substrate of E. coli TxrR, but instead it is a competitive inhibitor for the reduction of thioredoxin with a Kᵢ of 0.15 μM. E. coli mutants lacking a functional glutaredoxin system (glutathione reductase, GSH or glutaredoxin 1) were much more sensitive to inhibition by ebselen, which thereby will inhibit the essential enzyme ribonucleotide reductase (RNR) required for DNA synthesis. A main target of action of ebselen is the thioredoxin system. It follows that gram positive bacteria or other microorganisms lacking GSH will be particularly susceptible to ebselen. The present invention demonstrates that the well tolerated drug ebselen inhibits bacterial growth due to the large differences in structure and mechanism of the bacterial and mammalian thioredoxin reductases, establishing the drug as a novel chemotherapeutic principle.

It has been reported that ebselen inhibits bacteria growth with much higher sensitivity towards Staphylococcus aureus than E. coli. However the mechanism behind this inhibition was not previously known. The present inventors have found that ebselen and its diselenide are strong inhibitors of E. coli TrxR. In bacterial inhibition experiments using mutant strains lacking the enzyme glutathione reductase (GR encoded by the gor gene) or glutathione (gshA⁻ strain cannot synthesize GSH) showed increased sensitivity towards ebselen. The interaction mechanism of ebselen and its diselenide with E. coli was studied showing the formation of a relative stable ebselen-TrxR complex at the active site of the enzyme. Interestingly, we found that the sulfur analogue of ebselen, ebsulfur (PZ25), and its disulfide were not inhibitors of the E. coli enzyme, but rather were substrates for the E. coli TrxR. However, as shown below, this is not the case for all bacterial enzymes since the Helicobacter pylori TrxR is inhibited.

Comparing the kinetic parameters of the interaction between the compounds and the two enzyme systems, provides better understanding of the chemical basis for the inhibition mechanism of ebselen and its diselenide towards the E. coli TrxR. This enhanced understanding of the principle chemical mechanism of ebselen diverse activity towards mammalian and E. coli TrxR is very important for the use of the drug and also for the development of effective antibiotic drugs based on same mechanism. Furthermore, the finding that ebselen can inhibit E. coli TrxR leads us to a search for the new organoselenium compounds containing the basic structure of ebselen, to study their reactivity with E. coli thioredoxin reductase. We synthesized benzisoselenazol-3(2H)-ones and studied their reaction towards the thioredoxin reductase, to find out the relationship between the structure and reactivity. These compositions have, to varying extent, inhibitory effects on E. coli TrxR and bacterial growth, and therefore may be useful as antibiotics.

Different classes of benzisoselenazol-3(2H)-one compounds such as N-aryl (EbSe 7-10), N-unsubstituted (EbSe 6), N-alkyl (EbSe 2-4), N-2-pyridyl (EbSe 11 & 12) and N-4-pyridyl (EbSe 13) substituted benzisoselenazol-3(2H)-ones as well as bis-benzisoselenazol-3(2H)-ones (EbSe 14-16) were synthesized. Their inhibition effect on E. coli thioredoxin reductase (TrxR) was studied by thioredoxin dependent DTNB disulfide reduction assay in vitro. Detailed kinetic studies show that bis-benzisoselenazol-3(2H)-ones compounds (EbSe 14-16) inhibit TrxR at nanomolar concentrations while compounds EbSe 7-10, 12-13, 2-4 and parent ebselen, 2-phenyl-1,2-benzisoselenazol-3(2H)-one (EbSe 6) inhibit at micromolar concentrations. Other compounds did not inhibit E. coli TrxR. Tryptophan fluorescence measurements were carried out to follow the reaction of these compounds with reduced thioredoxin. Like ebselen, these compounds also rapidly oxidized reduced thioredoxin. Different classes of benzisoselenazol-3(2H)-one-aryl (EbSe 1-5), unsubstituted (EbSe 6), alkyl (EbSe 7-8), 2-pyridyl (EbSe 9 & 10) and 4-pyridyl (11) substituted benzisoselenazol-3(2H)-ones, bisbenzisoselenazol-3(2H)-ones (EbSe 12-14), 7-azabenzisoselenazol-3(2H)-one (EbSe 17), selenamide (EbSe 20) and bis(2-carbamoyl)phenyl diselenide (EbSe 21) have various levels of antibiotic activity, and for example inhibit bacterial (e.g., E. coli) thioredoxin reductase (TrxR). Detailed kinetic studies show that bisbenzisoselenazol-3(2H)-ones compounds (EbSe 12-14) inhibit TrxR at nanomolar concentrations while compounds EbSe 6, 2, 9, 11-13, 17, and parent ebselen, 2-phenyl-1,2-benzisoselenazol-3(2H)-one (EbSe 1) inhibit at micromolar concentrations. Like ebselen, these compounds also rapidly oxidized reduced thioredoxin. See, U.S. Pat. No. 8,592,468, US 2014/0088149; 2011/0288130; and 2009/0005422.

U.S. Pat. No. 8,592,468 discloses that benzisoselenazol-3(2H)-one and bisbenzisoselenazol-3(2H)-one derivatives were tested as potential E. coli TrxR inhibitors, Measured IC₅₀ and Kᵢ values (Table 1) indicate that the compounds EbSe 1-4, 10-14 are potent inhibitors for E. coli TrxR. The presence of covalent bond between selenium and nitrogen is important for the biological property of ebselen derivatives. The inhibition effect of selenamide (EbSe 20) was tested, which also possess direct Se—N bond. However, it has reduced inhibition effect than ebselen derivatives. Other derivatives EbSe 5-9 did not show significant inhibition on E. coli TrxR. The oxidation properties of benzisoselenazol-3(2H)-one derivatives on reduced E. coli Trx-(SH)₂ were studied. Ebselen is reported as superfast thioredoxin oxidant, and hence, used as the reference to compare the oxidant property of other compounds. The change of fluorescence intensity of 0.2 μM Trx-(SH)₂ by mixing with 0.2 μM benzisoselenazol-3(2H)-one show that all of the ebselen derivatives can oxidize the reduced Trx as the reference compound ebselen under identical conditions.

From the data shown in Table 1, it can be clearly seen that the substitution at the nitrogen atom of the benzisoselenazol-3(2H)-one ring has a significant effect on the inhibition of TrxR. The substitution of benzisoselenazol-3(2H)-one linked by alkyl chains (EbSe 12-14) has stronger inhibitory effect than unsubstituted (EbSe 6), alkyl (EbSe 7-8), aryl (EbSe 1-5), 2-pyridyl (EbSe 9-10) substituted ones, and also than compound EbSe 11 where the condensed benzene ring of benzisoselenazol-3(2H)-one is replaced by a pyridine ring. Compounds EbSe 12-14 show similar inhibitory effect irrespective of substitution at the second nitrogen atom and the number of alkyl chains between the two nitrogen atoms. From this observation it seems the second heteroatom nitrogen present in these compounds seems to important characteristic for their strong inhibition. Comparison of EbSe 6-8 show there is no inhibition when hydrogen is substituted by methyl (EbSe 6) or tert-butyl (EbSe 7) group. On the other hand comparison of between EbSe 1, 10 and 11 indicates that modification of 2-phenyl-1,2-benzisoselenazol-3(2H)-one to 2-pyridyl benzisoselenazol-3(2H)-one or 7-azabenzisoselenazol-3(2H)-one does not have significant effect. Also inhibition is not much affected by the substitution of phenyl group attached to the nitrogen of benzisoselenazol-3(2H)-one. Selenamide EbSe 20 has by far less inhibition effect than the ebselen derivatives though direct Se—N bond present. The presence of a five membered heterocyclic ring in addition to a direct Se—N bond in the ebselen structure seems essential for their biological activities.

Bacterial TrxR is potent target for antibiotics development, in particular for the bacteria lacking glutathione system. E. coli DHB4 strains wt, gshA⁻, gor⁻, oxyR⁻ were used as the model to test the antibiotics activity of these ebselen derivatives. The M ICs of these compounds were list in Table 1. Corresponding to the inhibition capacity of E. coli TrxR, ebselen derivatives EbSe 1-4 and EbSe 11-14 had strong ability to inhibit the bacterial growth. E. coli wt strain, strains gshA⁻ or gor⁻ which lost a functional glutathione system show more sensitive to ebselen derivatives EbSe 1-4 and 11, suggesting glutathione system play a critical roles in the protection of bacteria from these compound. Whereas, all these strains exhibited the same sensitivity to EbSe 12, 14. This observation was verified by the further GPx activity measurement of these compounds (Table 1). The compounds EbSe 1-4 and 11 can react with glutathione and then induce the consumption of NADPH. In contrast, no GPx activity was observed for compound EbSe 12.

The inhibition of mammalian TrxR and the cytotoxicity of these ebselen derivatives (Table 1) was also examined. Ebselen EbSe 12-14 was the strongest inhibitor for mammalian TrxR with a nanomolar inhibitory level, and also showed toxicity for mammalian cells. Ebselen EbSe 4 had some activity to inhibit mammalian TrxR, but it was one of the least reagent among these compounds. This result may be explained by the property that the compound is the best reagent to react with glutathione. The other ebselen derivatives EbSe 1-3 did not inhibit mammalian TrxR and were less toxic reagents for the mammalian cells.

Different classes of benzisoselenazol-3(2H)-one substituted compounds were found to exhibit different antibiotic properties because of their inhibition capacity on bacterial thioredoxin reductase. Generally, the -aryl, 2-pyridyl and 4-pyridyl substituted compounds possess a good inhibition ability to bacterial TrxR as well as the strong inhibition on bacterial growth and less toxicity. But the more substitution such as with chloro, carbono, or nitro substitution can alter antibiotic property. The Se—N bond the structure is essential for the inhibition of bacterial TrxR as well as the inhibition of bacteria. Benzisoselenazol-3(2H)-one-unsubstituted or alkyl substituted compounds do not have the ability to inhibit bacterial TrxR. Bisbenzisoselenazol-3(2H)-ones have the strong inhibition for bacteria but also have the strongest toxicity for the mammalian cells.

TABLE 1
Inhibition constants of ebselen derivatives on E. coli TrxR, mammalian TrxR, E. coli growth, glutathione
peroxidase activity and HEK 293T cells growth

| | | | | MIC | | | | | |
|---|---|---|---|---|---|---|---|---|---|
| | | | | for | MIC | MIC | | IC50 | |
| | | IC50 | | wild | for | for | | for | IC50 |
| | | for | Kᵢ for | type | gshA- | Gor- | | recombinant | for |
| | | E. coli | E. coli | DHB4 | DHB4 | DHB4 | Relative | rat | HEK |
| Compound | | TrxR | TrxR | E. coli | E. coli | E. coli | GPx | TrxR | 293T |
| Number | Structural Formula | (μM) | (μM) | (μM) | (μM) | (μM) | activity | (μM) | (μM) |
| EbSe 2 | 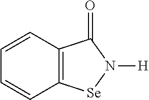 | 15 | N.D. 1.00 | | | | 0.97 | >10 | 95 |
| EbSe 3 | 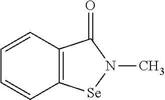 | 15 | N.D. | | | | 1.2 | >10 | 100 |
| EbSe 4 | 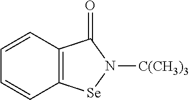 [SMILES: CC(C)(C)n1[se]c2ccccc2c1=O] | 15 | N.D. | | | | 1.1 | >10 | 75 |
| EbSe 5 | 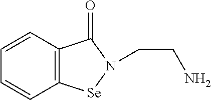 [SMILES: NCCn1[se]c2ccccc2c1=O] | 2.125 | 0.035 | | | | | | |
| EbSe 6 | 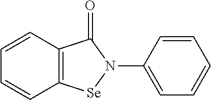 [SMILES: O=c1c2ccccc2[se]n1-c1ccccc1] | 6 | 0.30 | 40 | 26 | 15 | 1 | >10 | 120 |
| EbSe 7 | 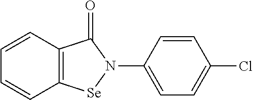 [SMILES: O=c1c2ccccc2[se]n1-c1ccc(Cl)cc1] | 7 | 0.55 | 34 | 20 | 23 | 0.64 | >10 | 120 |
| EbSe 8 | 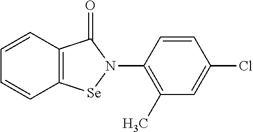 [SMILES: Cc1cc(Cl)ccc1-n1[se]c2ccccc2c1=O] | 6 | 0.25 | 47 | 13 | 24 | 0.67 | >10 | 80 |
| EbSe 9 | 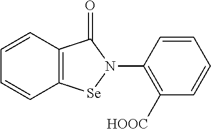 [SMILES: O=C(O)c1ccccc1-n1[se]c2ccccc2c1=O] | 7.5 | 1.20 | 49 | 24 | 31 | 2.1 | 0.8 | >160 |
| EbSe 10 | 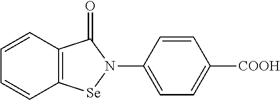 [SMILES: O=C(O)c1ccc(-n2[se]c3ccccc3c2=O)cc1] | 15 | N.D. | | | | 1.8 | >10 | 55 |
| EbSe 11 | 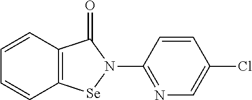 [SMILES: O=c1c2ccccc2[se]n1-c1ccc(Cl)cn1] | >40 | 0.3 N.D. | No Inh. | No Inh. | No Inh. | 0.87 | >10 | >160 |
| EbSe 12 | 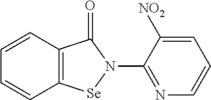 [SMILES: O=c1c2ccccc2[se]n1-c1ncccc1[N+](=O)[O-]] | 3 | 0.25 | No Inh. | No Inh. | No Inh. | 0.93 | >10 | 80 |
| EbSe 13 | 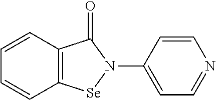 [SMILES: O=c1c2ccccc2[se]n1-c1ccncc1] | 3 | 1.5 | 45 | 21 | 24 | 1.4 | >10 | 60 |
| EbSe 14 | 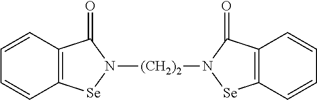 | 2 | 0.05 | 23 | 23 | 19 | No activity | 0.06 | 12.5 |
| EbSe 15 | 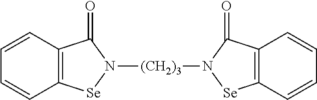 | 2.1 | 008 (0.038 for the Se rel. stock) | | | | | | 9.5 |
| EbSe 16 | 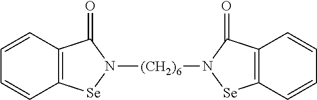 | 2.25 | 0.025 (0.01 for the Se rel. stock) | 20 | 20 | 35 | | | 9.2 |
| EbSe 19 | 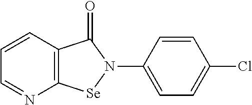 [SMILES: O=c1c2cccnc2[se]n1-c1ccc(Cl)cc1] | 3 | 0.5 | | | | | | |
| EbSe 22 | 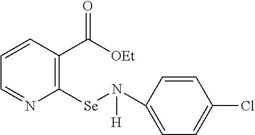 [SMILES: CCOC(=O)c1cccnc1[Se]Nc1ccc(Cl)cc1] | >10 | 7.5 | | | | | | |
| Dise1 | 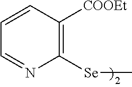 | >20 | No Inh. | No Inh. | No Inh. | No Inh. | 2.1 | | |
| 2 | 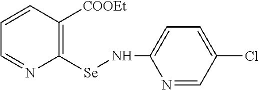 [SMILES: CCOC(=O)c1cccnc1[Se]Nc1ccc(Cl)cn1] | >10 | 7.5 | No Inh. | No Inh. | No Inh. | | | 60 |

Ebselen and ebselen diselenide are strong competitive inhibitors towards E. coli TrxR. When ebselen and ebselen diselenide are directly added in the solutions of E. coli TrxR and NADPH, no oxidations of NADPH were found. This is in line with the known fact that E. coli TrxR is strictly specific towards E. coli Trx. The effect of ebselen in the reduction of disulphide by E. coli Trx and TrxR was examined using both DTNB and insulin as substrates. Ebselen and its diselenide strongly inhibited the E. coli TrxR reduction towards E. coli Trx in a typical DTNB coupled assay. The same inhibition patterns are also shown for ebselen and ebselen diselenide in the insulin reduction assays (data not shown). E. coli Trx largely increases the rate of reduction of ebselen and ebselen diselenide by mammalian TrxR. Direct reduction of ebselen and the diselenide reduced E. coli Trx also were observed by fluorescence spectroscopy and the second-order rate constants were determined to be 2×10⁷ M⁻¹s⁻¹ and 1.7×10³ M⁻¹S⁻¹, respectively. Thus ebselen and the diselenide are targeting the E. coli TrxR rather than the E. coli Trx. The degree of inhibition caused by ebselen appears dependent on concentrations of Trx and ebselen. An increase in [Trx] at constant [EbSe] decreases the degree of inhibition and an increase in [EbSe] at constant [Trx] increases the degree of inhibition, showing a typical competitive inhibition towards the TrxR. A series of Lineweaver-Burk plots of the initial rate for the reduction of DTNB in the presence of ebselen and ebselen diselenide gave a typical pattern of competitive inhibitions. The dissociation constants Kᵢ for the ebselen-TrxR and ebselen diselenide-TrxR complexes derived from the slopes [(K_{M}/k_{cat})(1+[I]/Kᵢ)] were 0.14±0.05 μM and 0.46±0.05 μM, respectively.

TABLE 2
Kinetic parameters determined for ebselen, its diselenide and
their sulphur analogues with mammalian and E. coli TrxR.

| | Mammalian TrxR | | | E. coli TrxR | | |
|---|---|---|---|---|---|---|
| | k_{cat} | K_{M} | k_{cat}/K_{M} | k_{cat} | K_{M} | k_{cat}/K_{M} |
| Compounds | (min⁻¹) | (μM) | (μM⁻¹min⁻¹) | (min⁻¹) | (μM) | (μM⁻¹min⁻¹) |
| EbSe* | 588 | 2.5 | 235 | Inhibitor with Kᵢ = 0.15 ± 0.05 μM | | |
| (EbSe)₂* | 79 | 40 | 2 | Inhibitor with Kᵢ = 0.46 ± 0.03 μM | | |
| EbS | 1400 | 2.5 | 560 | 700 | 2.5 | 280 |
| (EbS)₂ | 1500 | 47 | 32 | 100 | 27.6 | 3.63 |

*from literature.

Ebselen inhibits the growth of E. coli strains and more sensitive towards gor⁻ and grxA⁻ mutants.

Since ebselen was a potent inhibitor of E. coli thioredoxin reductase we examined whether strains lacking components of the GSH-glutaredoxin reducing pathway would be more sensitive to the drug. Thus we examined the sensitivity of gor⁻ and gshA⁻ mutants to ebselen, which reside heavily on the TrxR reducing pathway. Wild type bacteria were more resistant than gor⁻ and gshA⁻ strains with gor⁻ and gshA⁻ strains being the most sensitive. This indicates that elimination of parts of the GSH pathway renders cells sensitive to ebselen. The explanation could be that ebselen inhibits TrxR or the thioredoxins, or is eliminated in cells by GSH. The sensitivity of strain trxA⁻C⁻ was similar, if not less, than that of the wild type, suggesting that the two E. coli thioredoxins were not primary targets for the compound. However ebselen may be affecting a thioredoxin 1 related function as the gshA⁻trxA⁻ strain was more sensitive to the compound. In rich LB liquid cultures, resistance could additionally be associated with GSH from the culture medium which binds and neutralizes ebselen. The sensitivity to ebselen was increased in minimal media where gor⁻ and gshA⁻ strains hardly grew in its presence.

Sensitivity of pathogenic bacteria to ebselen. Glutathione system is lacking and thus thioredoxin system is critical in many bacteria including some important pathogenic bacteria, such as methicillin resistant Staphylococcus aureus, Helicobacter pylori, Mycobacterium tuberculosis etc. Based on our principle that ebselen can target thioredoxin system in glutathione deficient bacteria, ebselen is the potential drug for inhibition of these bacterial. Methicillin resistant Staphylococcus aureus, Bacillus subtilis are quite sensitive to ebselen. We also investigated Mycobacterium tuberculosis sensitivity on ebselen, the test was done in the radiometric BACTEC 460 system. As shown in Table 3, several multidrug resistant Mycobacterium tuberculosis strains are sensitive to ebselen. The medium contains 5 g/l of albumin or 70 μM which will bind ebselen. Ebselen at 10 mg/I is 26 μM. The albumin free SH groups are about 50% or 35 μM. Therefore the MIC is dependent upon albumin saturation and probably lower than 20 mg/I.

Inhibition of ebselen was tested on H. pylori. For two macrolide sensitive strains, the minimal bactericidal concentration (MBC) are 3.125 and 6.25 μg/ml, for macrolide resistant strains, the MBC is 12.5 μg/ml. Taken together, our results strongly support that the inhibition of ebselen on these glutathione deficient bacteria is due to the oxidization of thioredoxin system by ebselen.

TABLE 3
Sensitivity of MDR Mycobacterium tuberculosis to ebselen

Sensitivity to ebselen (μg/ml)

| Strain | Ab-res | 80 | 40 | 20 | 10 |
|---|---|---|---|---|---|
| H37Rv | S | S | S | S | R |
| Panel3:24 | MDR | S | S | S | R |
| BTB 98-310 | MDR | S | S | S | R |

S: sensitive to rifampicin as positive control (no growth);
R: resistant.

TABLE 4
Bactericidal effects of ebselen on Helicobacter pylori

| | Sensitivity to | Sensitivity to ebselen (μg/ml) | | | | | | | |
|---|---|---|---|---|---|---|---|---|---|
| Strain | Macrolide | 100 | 50 | 25 | 12.5 | 6.25 | 3.125 | 1.56 | 0.78 |
| MS G6 | S | S | S | S | S | S | S | R | R |
| MS G142 | S | S | S | S | S | S | R | R | R |
| MR G162 | R | S | S | S | S | R | R | R | R |
| MR G193 | R | S | S | S | S | R | R | R | R |

S: sensitive;
R: resistant.

E. coli TrxR inhibition by ebselen derivatives. All the benzisoselenazol-3(2H)-one and bisbenzisoselenazol-3(2H)-one derivatives were tested as potential E. coli TrxR inhibitors by standard DTNB assay. IC₅₀ values were calculated by following the activity of TrxR reducing DTNB by NADPH at 412 nm. The reactions were started by adding 1 mM DTNB to the mixture of 100 nM TrxR, 2 μM Trx, 240 μM NADPH, and different concentration of inhibitor (1-40 μM). For determining the inhibition constants (Kᵢ), indicated amount of inhibitor was mixed with total volume 500 μL containing 1 mM DTNB, 240 μM NADPH, fixed thioredoxin concentration (1 or 2 or 4 μM) and buffer containing 50 mM Tris-Cl, 2 mM EDTA, pH 7.5. The reactions were started by adding 6 nM TrxR at room temperature. Inhibition constants (Kᵢ) for all the compounds were measured from Dixon plot, which plots 1/v versus [I]=A₄₁₂/min, I=Inhibitor concentration). Measured IC₅₀ and Kᵢ values (Table 1) indicate that the compounds EbSe 6-9, 12-16 are potent inhibitors for E. coli TrxR. The presence of covalent bond between selenium and nitrogen is so important for the biological property of ebselen derivatives. Other derivatives did not show significant inhibition on E. coli TrxR.

Oxidation E. coli Trx-(SH)₂ by ebselen derivatives. Oxidant property of benzisoselenazol-3(2H)-one derivatives on reduced E. coli Trx-(SH)₂ were studied by fluorescence spectroscopy. This property was chosen to follow the reaction of Trx with benzisoselenazol-3(2H)-one derivatives since E. coli Trx-(SH)₂ has 3-fold higher tryptophan fluorescence than Trx-S₂. Ebselen is reported as superfast thioredoxin oxidant[32] and hence, used as the reference to compare the oxidant property of other compounds. The change of fluorescence intensity of 0.2 μM Trx-(SH)₂ by mixing with 0.2 μM benzisoselenazol-3(2H)-one show that they all can oxidize the reduced Trx as the reference compound ebselen under identical conditions.

Correlation between the structure and their inhibition. From the data shown in Table 1, it can be clearly seen that the substitution at nitrogen atom of benzisoselenazol-3(2H)-one ring have significant effect on the inhibition of TrxR. The substitution of benzisoselenazol-3(2H)-one linked by alkyl chains (EbSe 14-16) has stronger inhibitory effect than unsubstituted (EbSe 6), alkyl (EbSe 2-4), aryl (EbSe 7-10), 2-pyridyl (EbSe 11-12) and 4-pyridyl (EbSe 13) substituted ones. Compounds EbSe 14-16 show similar inhibitory effect irrespective of substitution at the second nitrogen atom and the number of alkyl chains between the two nitrogen atoms. From this observation it seems the second heteroatom nitrogen present in these compounds seems to important characteristic for their strong inhibition. Comparison of EbSe 2-4 show there is no inhibition when hydrogen is substituted by methyl (6) or tert-butyl (7) group. On the other hand comparison of EbSe 6, 12 and 13 indicates that modification of the 2-phenyl-1,2-benzisoselenazol-3(2H)-one into an N-2-pyridyl benzisoselenazol-3(2H)-one or an N-4-pyridyl benzisoselenazol-3(2H)-one does not have a significant effect. Also inhibition is not much affected by the substitution of phenyl group attached to the nitrogen of benzisoselenazol-3(2H)-one.

Inhibition of bacterial growth by ebselen derivatives. Bacterial TrxR is potent target for antibiotics development, in particular for the bacteria lacking glutathione system. Here E. coli DHB4 strains wt, gshA⁻, gor⁻, oxyR⁻ were used as the model to test the antibiotics activity of these ebselen derivatives corresponding to the inhibition capacity of E. coli TrxR, ebselen derivatives EbSe 6-9 and 13-16 had strong ability to inhibit the bacterial growth. E. coli wt strain, strains gshA⁻ or gor⁻ which lost a functional glutathione system show more sensitive to ebselen derivatives EbSe 6-9 and 13, suggesting glutathione system play a critical roles in the protection of bacteria from these compound. Whereas, all these strains exhibited the same sensitivity to EbSe 14, 16.

Inhibition of H. pylori TrxR and H. pylori strains by PZ-25 (ebsulfur). H. pylori TrxR activity was inhibited by 4, 20, and 40 μM of PZ-25 by insulin reduction assay. Consistent with the inhibition of H. pylori TrxR activity, H. pylori strains were shown to be sensitive to ebsulfur. For NCTC11637 strain, the MIC for ebselen, PZ-25, metronidazole was 3.13, 1.56, and 0.78 μg/ml respectively. For strain YS-16, The MIC for ebselen, PZ-25, metronidazole was 3.13, 0.39, 6.25 μg/ml respectively.

Ebselen is an antioxidant due to the special selenium chemistry it interplayed with thiol and hydrogen peroxide. The mechanism was recently described to be via the mammalian thioredoxin system with the formation of ebselen diselenide as an important part of the mechanism. Ebselen also has low toxicity for the human body because the selenium moiety is not liberated during biotransformation so it does not enter the selenium metabolism of the organism. At low concentrations, ebselen even inhibits a number of enzymes involved in inflammation such as lipoxygenases, NO synthesase, protein kinase C and H⁺/K⁺-ATPase. The inhibitions were manifested on the cellular level and may contribute to the anti-inflammatory potential of ebselen. Ebselen has another interesting pharmaceutical profile, namely its antibacterial character, targeting the bacterial thioredoxin reductase as shown herein, with structure and properties distinct from the mammalian counterpart. The inhibition kinetic parameters determined for the ebselen and its diselenide towards E. coli TrxR indicate that both compounds are strong inhibitors with nanomolar affinities. It was reported that the growth of Staphylococcus aureus 209P was inhibited by 0.20 μg/ml of ebselen, while strains of the family Enterobacteriaceae were more resistant to the drug. The selenium in PZ51 was essential, since its sulfur analogue (PZ25) lost the antibacterial activity. In cell experiments, ebselen clearly inhibited bacterial strains. The mutants lacking glutathione reductase (gor⁻) and glutathione (gshA⁻) showed increased sensitivity.

In E. coli, it was long proposed that thioredoxin system and glutaredoxin system are two crucial pathways for the electron flow to be delivered to the ribonucleotide reductase for DNA synthesis. Thiol reductions by the two systems also play key roles in cell growth as well as redox regulation of a variety of biological functions. The sensitivity to ebselen increased with mutants lacking glutathione reductase (gor⁻) and glutathione (gshA⁻), indicating that perturbations of the GSH reducing pathway render cells more sensitive to ebselen. The sensitivity to ebselen was increased in minimal media where gor⁻ and gshA⁻ strains hardly grew. The increased sensitivity in minimal media could be expected since lack of GSH would increase demands for electrons from the thioredoxin system for sulfate reduction. The results clearly show that elimination of GSH or glutathione reductase which makes cells more dependent on the thioredoxin system leads to a greater degree of inhibition. From the results previously published, the large difference in sensitivity of bacteria to ebselen is clearly correlated to having GSH or not. Gram positive strains of bacteria like S. aureus or B. subtillus lack GSH. Bacillus subtilis e.g. has formally no glutaredoxin pathway but several thioredoxins which are essential. The bacterial thioredoxin reductases are therefore drug targets for ebselen.

From a simple chemical point of view, the reaction of Ebselen with the E. coli TrxR is much slower or completely stopped for the reasons of a highly polar CysS-SeEb bond in the second disulphide interchange reaction. E. coli TrxR undergoes an essential conformation change allowing electron flows to go through from NADPH to FAD and the active disulphide in each catalytic cycle. The kinetic constant of this conformation change is ˜53 s⁻¹ at 25° C. The inhibition of the E. coli TrxR by ebselen and its diselenide are therefore believed to result from the slow release of ebselen selenol from the relatively polar selenenolsulfide bridge, and the determined conformation change from FR to FO of the E. coli TrxR-SeEbSe complex.

The E. coli TrxR is known for its high specificity towards its Trx, and in fact, PZ25 and its disulphide are the first two small molecules found as substrates. The specificity of E. coli TrxR as compared with its mammalian counterpart may be principally attributed to this specific conformation change, which differentiates between substrate oxidants except where their disulphide exchange reactions with the active-site thiols in the E. coli TrxR are fast enough to not disrupt the normal conformation change of the enzyme.

The drug has no inhibitory activity of mammalian thioredoxin reductases due to their highly different structures and mechanisms when compared with the ubiquitous bacterial enzymes. The ebselen molecule is thus an antioxidant drug with useful antibacterial spectrum and two effects for the price of one.

Thus the non-toxic drug ebselen inhibits bacterial growth due to the large differences in its mechanism of action towards bacterial and mammalian TrxR, the two structurally very distinct enzymes. In pathogenic bacteria like M. tuberculosis the defense from the bacterium against the host killing by reactive oxygen species derived from macrophages is dependent on thioredoxin coupled peroxidases. Thus the inhibition of the thioredoxin system would also sensitize the bacteria in the intracellular environment. Therefore ebselen and derivatives would be effective agents against the survival and virulence of M. tuberculosis in its dormant stage in macrophages where the pathogen has to defend itself against reactive oxygen species from the host as well as to repair its DNA. The latter process is dependent on the thioredoxin system and ribonucleotide reductase and targeted by ebselen. In fact ebselen is also an effective direct inhibitor of E. coli ribonucleotide reductase (data not shown). Different classes of benzisoselenazol-3(2H)-one substituted compounds were found to exhibit different antibiotic properties because of their inhibition capacity on bacterial thioredoxin reductase. Generally, the N-aryl, N-2-pyridyl and N-4-pyridyl substituted compounds as well as bis-benzisoselenazol-3(2H)-ones possess a good inhibition ability towards bacterial TrxR. But substitution with chloro, carboxy, or nitro groups can alter the antibiotic properties.

### SUMMARY OF THE INVENTION

Unless defined otherwise, all technical and scientific terms used herein have the same meaning as commonly understood by one of the ordinary skill in the art to which this invention belongs. Although any methods and materials similar or equivalent to those described herein can be used in the practice or testing of the compositions or unit doses herein, some methods and materials are now described. Unless mentioned otherwise, the techniques employed or contemplated herein are standard methodologies. The materials, methods and examples are illustrative only and not limiting. The details of one or more inventive embodiments are set forth in the claims and the description herein. Other features, objects, and advantages of the inventive embodiments disclosed and contemplated herein can be combined with any other embodiment unless explicitly excluded. Unless otherwise indicated, open terms for example “contain,” “containing,” “include,” “including,” and the like mean comprising. The singular forms “a”, “an”, and “the” are used herein to include plural references unless the context clearly dictates otherwise. Accordingly, unless the contrary is indicated, the numerical parameters are approximations that may vary depending upon the desired properties sought to be obtained by the present invention, and the criticality of the quantitative expression. Unless otherwise indicated, some embodiments herein contemplate numerical ranges. When a numerical range is provided, unless otherwise indicated, the range includes the range endpoints. The term “about”, for a non-critical quantitative value or range, can refer to a numerical value of ±0.5 log 2 of the referenced value, i.e., for a value of 1, an implied range of 0.71-1.41.

The term “derivative” can include one or more conformational isomers (e.g., cis and trans isomers) and all optical isomers (e.g., enantiomers and diastereomers), racemic, diastersomeric and other mixtures of such isomers, as well as solvates, hydrates, isomorphs, polymorphs, tautomers, esters, salt forms, and prodrugs, which otherwise meet specified functional criteria. By “tautomers” is meant chemical compounds that may exist in two or more forms of different structure (isomers) in equilibrium, the forms differing, usually, in the position of a hydrogen atom. Various types of tautomerism can occur, including keto-enol, ring-chain and ring-ring tautomerism. The expression “prodrug” refers to compounds that are drug precursors which following administration, release the drug in vivo via some chemical or physiological process (e.g., a prodrug on being brought to the physiological pH or through enzyme action is converted to the desired drug form).

The present invention provides a combination therapy for diseases characterized by chronic inflammatory response and oxidative stress comprising reduced glutathione and a isoselenazol or isothiazol derivative, e.g., ebselen (PZ-51) or its sulfur analog ebsulfur (PZ-25). Such conditions typically require long term therapy, and as such, oral dosage forms are preferred. Likewise, for convenience, it is preferred that the oral dosage form contain both components.

Glutathione is absorbed in the first part of the ileum, generally before the ligament of Treitz, and therefore delayed release or bioavailability is to be avoided. On the other hand, there is a significant risk of drug interaction, both within the dosage form, and in the stomach, if the two components are not physically separated. Therefore, a preferred embodiment of the invention provides a two or more component oral dosage form pharmaceutical formulation comprising a rapidly dissolving capsule containing powdered reduced L-glutathione mixed with crystalline ascorbic acid, e.g., in a 1:2 molar ratio, and within the same capsule, enteric release granules of the isoselenazol or isothiazol derivative, designed to release the active drug 2-6 hours after administration. The formulation is preferably administered on an empty stomach, to facilitate glutathione bioavailability. See, PCT/US97/23879; US 20050222046; US 20020136763; U.S. Pat. Nos. 6,896,899; 6,586,404; 6,423,487; 6,350,647; 6,204,248; 6,159,500; 5,326,757; 5,204,114; 4,454,125; 8,592,468; 7,671,211.

Isoselenazol or Isothiazol Derivatives

The isoselenazol or isothiazol derivative may have anti-inflammatory activity. See, Vincent Galet, Jean-Luc Bernier, Jean-Piere Henichart, Daniel Lesieur, Claire Abadie, Luc Rochette, Albert Lindenbaum, Jacqueline Chalas, Jean-Francois Renaud de la Faverie, “Benzoselenazolinone Derivatives Designed To Be Glutathione Peroxidase Mimetics Feature Inhibition of Cyclooxygenase/5-Lipoxygenase Pathways and Anti-inflammatory Activity”, J. Med. Chem., 1994, 37 (18), pp 2903-2911, DOI: 10.1021/jm00044a011; Zade, Sanjio S., et al. “Convenient Synthesis, Characterization and GPx-Like Catalytic Activity of Novel Ebselen Derivatives.” European Journal of Organic Chemistry 2004.18 (2004): 3857-3864; Sarma, Bani Kanta, and Govindasamy Mugesh. “Antioxidant Activity of the Anti-Inflammatory Compound Ebselen: A Reversible Cyclization Pathway via Selenenic and Seleninic Acid Intermediates.” Chemistry-A European Journal 14.34 (2008): 10603-10614; Bosch-Morell, Francisco, et al. “Efficacy of the antioxidant ebselen in experimental uveitis.” Free Radical Biology and Medicine 27.3 (1999): 388-391; Leyck, S., and M. J. Parnham. “Acute antiinflammatory and gastric effects of the seleno-organic compound ebselen.” Agents and actions 30.3-4 (1990): 426-431.

Isoselenazol or isothiazol derivatives generally have the Formula I (and include ebselen itself), and pharmaceutically acceptable salts thereof:

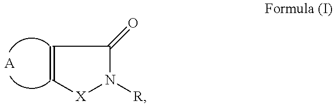

wherein X is selenium or sulfur, and optionally adducts of the selenium or sulfur (which may be active or prodrugs for the active derivative); with the proviso that X can be S in the resulting derivative if it is an inhibitor of prokaryotic thioredoxin reductase having an IC₅₀ of less than about 25 μM, and wherein R is hydrogen or an organic moiety selected from the group consisting of: (a) alkyl having a carbon chain of 1 to 14 carbon atoms wherein the carbon chain is branched or unbranched which is optionally substituted with bensisoselenazol-3(2H)-one-2-yl, benzisotiazol-3(2H)-one-2-yl, OH, alkoxyl, SH, NH₂, N-alkylamino, N,N-dialkylamino, COOH, aryl which is optionally substituted with C₁-C₅ alkyl, OH, alkoxyl, SH, NH₂, N-alkylamino, N,N-dialkylamino, COOH, CHO, NO₂, F, Cl, Br, I, or heteroaryl which is optionally substituted with C₁-C₅ alkyl, OH, alkoxyl, SH, NH₂, N-alkylamino, N,N-dialkylamino, COOH, CHO, NO₂, F, Cl, Br, or I, (b) aryl which is optionally substituted with C₁-C₅ alkyl, OH, alkoxyl, SH, NH₂, N-alkylamino, N,N-dialkylamino, COOH, CHO, NO₂, F, Cl, Br, or I, (c) heteroaryl which is optionally substituted with C₁-C₅ alkyl, OH, alkoxyl, SH, NH2, N-alkylamino, N,N-dialkylamino, COOH, CHO, NO₂, F, Cl, Br, or I, (d) wherein A represents an organic ring structure, for example a saturated, unsaturated or polyunsaturated 3 to 6 member carbon chain and wherein N may optionally substitute for one or more carbons, and which is optionally substituted with one or more of OR, SR, or alkylamino, C₁-C₅ alkyl, OH, alkoxyl, SH, NH₂, N-alkylamino, N,N-dialkylamino, COOH, CHO, NO₂, F, Cl, Br, or I. The composition may further include an isothiazoline derivative. See, U.S. Pat. Nos. 8,496,952, 5,364,649, and 4,150,026. The ebselen derivative may be selected from the group consisting of EbSe2, EbSe3, EbSe4, EbSe5, EbSe6, EbSe7, EbSe8, EbSe9, EbSe10, EbSe11, EbSe12, EbSe13, EbSe14, EbSe15, EbSe16, and EbSe19. EbSe11 has a higher IC₅₀ than the other compounds, and therefore is not preferred on at least that basis.

As noted above, in some cases, the selenium of ebselen may be replaced with sulfur, resulting in an ebsulfur derivative. As discussed below, most such compositions are ineffective to meet the criteria associated with an efficacious therapy. However, at least ebsulfur-23, the sulfur analog of ebselen-10, has certain promising characteristics.

Ebselen is a strong and irreversible inhibitor of rabbit lipooxygenase, an effect which is blocked by large concentrations of GSH or other thiols. Walther, Matthias, et al. “The inhibition of mammalian 15-lipoxygenases by the anti-inflammatory drug ebselen: dual-type mechanism involving covalent linkage and alteration of the iron ligand sphere.” Molecular pharmacology 56.1 (1999): 196-203.

The Isoselenazol or isothiazol derivative is preferably an antibiotic which inhibits bacterial thioredoxin reductase.

In a typical clinical setting, an isoselenazol or isothiazol derivative will be administered as a standard dose (range of mg/kg or mg/m³), having a standard repetition (once, twice, three times, four times, etc., per day). Therefore, the range of concentrations will generally be defined by the standard dosage forms, though the patient size may also be calculated to achieve a better estimate.

The invention also provides a pharmaceutical composition comprising the benzisoselenazolonyl or benzisothiazolonyl derivatives or their pharmaceutically acceptable salts and pharmaceutically acceptable excipient or carrier, in combination with stabilized reduced glutathione. The two components may be chemically isolated in both the dosage form during administration and after administration during absorption.

For example, the composition can be used in the form of tablet, suppository, pill, soft and hard gelatin capsule, granule, solution, suspension or aerosol. Preferred is a single orally administered unit dosage form which combine a rapid release glutathione capsule with delayed release benzisoselenazolonyl or benzisothiazolonyl derivative granules. The pharmaceutical composition may include conventional excipients or carriers. The glutathione component of the formulation is preferably a highly concentrated, charge transfer complex of the glutathione and e.g., crystalline ascorbic acid. The ascorbic acid acts as a sacrificial antioxidant for the glutathione, and also neutralizes the charge on the glutathione powder to form a densified mixture. The charge transfer complex further is believed to enhance absorption of the glutathione.

The composition may additionally contain other therapeutic agents and the like.

Ebselen is a crystalline solid which is not directly soluble in aqueous solution. In some embodiments, the pharmaceutically acceptable composition is a storage-stable article includes from 0.01 to 20 percent by weight of the controlled-release coating, which can include beeswax, beeswax and glyceryl monostearate, shellac and cellulose, cetyl alcohol, mastic and shellac, shellac and stearic acid, polyvinyl acetate and ethyl cellulose, neutral copolymer of polymethacrylic acid ester (Eudragit L30D), copolymer of methacrylic acid and methacrylic acid methylester (Eudragit S), neutral copolymers of polymethacrylic acid esters containing metallic stearates, and/or neutralized hydroxypropyl methylcellulose phthalate polymer.

In one embodiment, the benzisoselenazolonyl or benzisothiazolonyl derivatives are formed into coated granules. These granules may be formed by, e.g., agglomeration, air suspension chilling, air suspension drying, balling, coacervation, comminution, compression, pelletization, cryopelletization, extrusion, granulation, homogenization, inclusion complexation, lyophilization, melting, mixing, molding, pan coating, solvent dehydration, sonication, spheronization, spray chilling, spray congealing, spray drying, or other processes known in the art.

Various embodiments of the invention, as described in more detail below, include a surfactant. A surfactant may be used to facilitate dissolution of the ebselen or ebsulfur derivative, especially where the ebselen or ebsulfur derivative is micronized, i.e., finely divided into small particles, such as 1-25 micron diameter. The particles preferably have high surface area (>15% more than a corresponding sphere), and are separated in a hygroscopic medium, such as starch. As a result, after the granules pass through the stomach, the pH changes to near neutral or slightly basic, and the delayed-release coating begins to dissolve. As the “hull” is breached, water is drawn into the interstices of the granule, and the starch expands and mechanically disintegrates the granule. The surfactant ensures that the micronized particles are wetted, and the ebselen or ebsulfur derivative then dissolves in the aqueous medium. The delayed release may be prolonged, to provide an extended release profile, by controlling the size of the granules, the delayed release coating, the amount and nature of the disintegrant, the amount and nature of the surfactant (if provided), the size and nature of the particles, and other known variables. The release provide of the ebselen or ebsulfur derivative is preferably provided to achieve, on one hand, a blood concentration which maintains therapeutically effective concentrations, while optionally also achieving peak levels that may have enhanced antimicrobial activity. Preferably, where antimicrobial activity is pertinent, the maintained level is above the minimum inhibitory concentration for the target strain.

Hydrophilic surfactants can be used to provide any of several advantageous characteristics to the compositions, including: increased solubility of the active ingredient in the solid carrier, improved dissolution of the active ingredient; improved solubilization of the active ingredient upon dissolution; enhanced absorption and/or bioavailability of the active ingredient, particularly a hydrophilic active ingredient; and improved stability, both physical and chemical, of the active ingredient. The hydrophilic surfactant can be a single hydrophilic surfactant or a mixture of hydrophilic surfactants, and can be ionic or non-ionic. See US 20150273067. Likewise, various embodiments of the invention include a lipophilic component, which can be a lipophilic surfactant, including a mixture of lipophilic surfactants, a triglyceride, or a mixture thereof. The lipophilic surfactant can provide any of the advantageous characteristics listed above for hydrophilic surfactants, as well as further enhancing the function of the surfactants. See, U.S. Pat. No. 5,985,319; www.fda.gov/downloads/Drugs/ . . . /Guidances/UCM070640.pdf. Surfactants can be any surfactant suitable for use in pharmaceutical compositions. Suitable surfactants can be anionic, cationic, zwitterionic or non-ionic.

Although polyethylene glycol (PEG) itself does not function as a surfactant, a variety of PEG-fatty acid esters have useful surfactant properties. Polyethylene glycol (PEG) fatty acid diesters are also suitable for use as surfactants. A large number of surfactants of different degrees of lipophilicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Polyglycerol esters of fatty acids, esters of propylene glycol and fatty acids, mixtures of propylene glycol fatty acid esters and glycerol fatty acid esters, mono- and diglycerides, sterols and derivatives of sterols, PEG-sorbitan fatty acid esters, ethers of polyethylene glycol and alkyl alcohols, esters of sugars, hydrophilic PEG-alkyl phenol, polyoxyethylene-polyoxypropylene block copolymers, sorbitan esters of fatty acids, esters of lower alcohols (C₄ to C₁₄) and fatty acids (C₈ to C₁₈), free fatty acids, particularly C₆₋₂₂ fatty acids, and bile acids, are available surfactants. In general, surfactants or mixtures of surfactants that solidify or are solid at ambient room temperature are most preferred.

IONIC SURFACTANTS, including cationic, anionic and zwitterionic surfactants, are available. hydrophilic surfactants: anionic surfactants include fatty acid salts and bile salts; cationic surfactants include carnitines. Specifically, ionic surfactants may include sodium oleate, sodium lauryl sulfate, sodium lauryl sarcosinate, sodium dioctyl sulfosuccinate, sodium cholate, sodium taurocholate; lauroyl carnitine; palmitoyl carnitine; and myristoyl carnitine. Fatty acids are typically sodium salts, though other cation counterions can also be used, such as alkali metal cations or ammonium. These surfactants include: Sodium caproate, Sodium caprylate, Sodium caprate, Sodium laurate, Sodium myristate, Sodium myristolate, Sodium palmitate, Sodium palmitoleate, Sodium oleate, Sodium ricinoleate, Sodium linoleate, Sodium linolenate, Sodium stearate, Sodium lauryl sulfate (dodecyl), Sodium tetradecyl sulfate, Sodium lauryl sarcosinate, Sodium dioctyl sulfosuccinate [sodium docusate (Cytec)]. BILE SALTS Sodium cholate, Sodium taurocholate, Sodium glycocholate, Sodium deoxycholate, Sodium taurodeoxycholate, Sodium glycodeoxycholate, Sodium ursodeoxycholate, Sodium chenodeoxycholate, Sodium taurochenodeoxycholate, Sodium glyco chenodeoxycholate, Sodium cholylsarcosinate, Sodium N-methyl taurocholate. PHOSPHOLIPIDS Egg/Soy lecithin [Epikuron® (Lucas Meyer), Ovothin® (Lucas Meyer)], Cardiolipin, Sphingomyelin, Phosphatidylcholine, Phosphatidyl ethanolamine, Phosphatidic acid, Phosphatidyl glycerol, Phosphatidyl serine. PHOSPHORIC ACID ESTERS Diethanolammonium polyoxyethylene-oleyl ether phosphate, Esterification products of fatty alcohols or fatty alcohol ethoxylates with phosphoric acid or anhydride. CARBOXYLATES Ether carboxylates (by oxidation of terminal OH group of fatty alcohol ethoxylates), Succinylated monoglycerides [LAMEGIN ZE (Henkel)], Sodium stearyl fumarate, Stearoyl propylene glycol hydrogen succinate, Mono/diacetylated tartaric acid esters of mono- and diglycerides, Citric acid esters of mono-, diglycerides, Glyceryl-lacto esters of fatty acids (CFR ref. 172.852), Acyl lactylates, lactylic esters of fatty acids, calcium/sodium stearoyl-2-lactylate, calcium/sodium stearoyl lactylate, Alginate salts, Propylene glycol alginate. SULFATES AND SULFONATES Ethoxylated alkyl sulfates, Alkyl benzene sulfones, .alpha.-olefin sulfonates, Acyl isethionates, Acyl taurates, Alkyl glyceryl ether sulfonates, Octyl sulfosuccinate disodium, Disodium undecylenamideo-MEA-sulfosuccinate. CATIONIC SURFACTANTS Hexadecyl triammonium bromide, Dodecyl ammonium chloride, Alkyl benzyldimethylammonium salts, Diisobutyl phenoxyethoxydimethyl benzylammonium salts, Alkylpyridinium salts, Betaines (trialkylglycine), Lauryl betaine (N-lauryl,N,N-dimethylglycine), Ethoxylated amines, Polyoxyethylene-15 coconut amine NON-IONIC HYDROPHILIC SURFACTANTS include alkylglucosides; alkylmaltosides;

alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols with fatty acids, glycerides, vegetable oils, hydrogenated vegetable oils, and sterols; sugar esters; sugar ethers; sucroglycerides; polyethoxylated fat-soluble vitamins or derivatives; and mixtures thereof. The hydrophilic surfactant can also be, or can include as a component, an ionic surfactant, such as alkyl ammonium salts; bile acids and salts, analogues, and derivatives thereof; fusidic acid and derivatives thereof; fatty acid derivatives of amino acids, oligopeptides, and polypeptides; glyceride derivatives of amino acids oligopeptides, and polypeptides; acyl lactylates; mono- and di-acetylated tartaric acid esters of mono- and di-glycerides; succinylated monoglycerides; citric acid esters of mono- and di-glycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof. Ionic surfactants include lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidyiglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono- and di-acetylated tartaric acid esters of mono- and di-glycerides, citric acid esters of mono- and di-glycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof. As with the hydrophilic surfactants, lipophilic surfactants can be reaction mixtures of polyols and fatty acids, glycerides, vegetable oils, hydrogenated vegetable oils, and sterols.

A composition disclosed herein, e.g., a substrate granule of a delayed release component compositions can be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A powder constitutes a finely divided (milled, micronized, nanosized, precipitated) form of an active ingredient or additive molecular aggregates or a compound aggregate of multiple components or a physical mixture of aggregates of an active ingredient and/or additives. Such substrates can be formed of various materials known in the art, such as, for example: sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapatite, tricalcium phosphate, talc, or titania; and polyols, such as mannitol, xylitol, sorbitol or cyclodextrin. The substrate can also be formed of any of the active ingredients, surfactants, triglycerides, solubilizers or additives described herein. In one particular embodiment, the substrate is a solid form of an additive, an active ingredient, a surfactant, or a triglyceride; a complex of an additive, surfactant or triglyceride and an active ingredient; a coprecipitate of an additive, surfactant or triglyceride and an active ingredient, or a mixture thereof. The solid pharmaceutical compositions can optionally include one or more additives, sometimes referred to as excipients. The additives can be contained in an encapsulation coat in compositions, which include an encapsulation coat, or can be part of the solid carrier, such as coated to an encapsulation coat, or contained within the components forming the solid carrier. Alternatively, the additives can be contained in the pharmaceutical composition but not part of the solid carrier itself. Specific, non-limiting examples of additives are described below.

Solubilizers include: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropylmethyl cellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, .delta.-valerolactone and isomers thereof, .beta.-butyrolactone and isomers thereof; and other solubilizers known in the art, such as dimethyl acetamide, dimethyl isosorbide (Arlasolve DMI (ICl)), N-methyl pyrrolidones (Pharmasolve (ISP)), monooctanoin, and diethylene glycol monoethyl ether (available from Gattefosse under the trade name Transcutol).

Excipients may also be employed, including but not limited to: binders (adhesives), i.e., agents that impart cohesive properties to powdered materials through particle-particle bonding, such as matrix binders (dry starch, dry sugars), film binders (PVP, starch paste, celluloses, bentonite, sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose); diluents or fillers, such as lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose; disintegrants or super disintegrants, such as croscarmellose sodium, starch, starch derivatives, clays, gums, cellulose, cellulose derivatives, alginates, crosslinked polyvinylpyrrolidone, sodium starch glycolate and microcrystalline cellulose.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. The amounts of such additives can be readily determined by one skilled in the art, according to the particular properties desired.

The delayed release component of the pharmaceutical composition and/or the solid carrier particles can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be effected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

An “extended release coating” is a coating designed to effect delivery over an extended period of time. Preferably, the extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness. An “enteric coating” is a mixture of pharmaceutically acceptable excipients which is applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: resistance to dissolution and disintegration in the stomach; impermeability to gastric fluids while in the stomach; ability to dissolve or disintegrate rapidly at the target intestine site; physical and chemical stability during storage; and non-toxicity.

“Delayed release” refers to the delivery so that the release can be accomplished at some generally predictable location in the lower intestinal tract more distal to that which would have been accomplished if there had been no delayed release alterations. The preferred method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. The polymers may be anionic carboxylic polymers, e.g.: shellac (not preferred, insect derived); acrylic polymers, varying performance based on the degree and type of substitution, e.g., methacrylic acid copolymers and ammonia methacrylate copolymers. The Eudragit series L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for extended release. The Eudragit series L, L-30D and S are insoluble in stomach and dissolve in the intestine; cellulose derivatives, e.g., ethyl cellulose; reaction mixtures of partial acetate esters of cellulose with phthalic anhydride; performance varies based on the degree and type of substitution. Cellulose acetate phthalate (CAP) dissolves in pH>6. Aquateric (FMC) is an aqueous based system and is a spray dried CAP psuedolatex with particles<1 μm. Other components in Aquateric can include pluronics, Tweens, and acetylated monoglycerides; cellulose acetate trimellitate (Eastman); methylcellulose (Pharmacoat, Methocel); hydroxypropylmethyl cellulose phthalate (HPMCP). The performance can vary based on the degree and type of substitution. HP-50, HP-55, HP-55S, HP-55F grades are suitable; hydroxypropylmethyl cellulose succinate (HPMCS; AQOAT (Shin. Etsu)). The performance can vary based on the degree and type of substitution. Suitable grades include AS-LG (LF), which dissolves at pH 5, AS-MG (MF), which dissolves at pH 5.5, and. AS-HG (HF), which dissolves at higher pH. These polymers are offered as granules, or as fine powders for aqueous dispersions; Poly Vinyl Acetate Phthalate (PVAP). PVAP dissolves in pH>5, and it is much less permeable to water vapor and gastric fluids; and Cotteric (by Colorcon). Combinations of the various materials disclosed herein, and multiple layers with corresponding sequential functions can also be used.

The coating can, and usually does, contain a plasticizer and possibly other coating excipients such as colorants, talc, and/or magnesium stearate, which are well known in the art. Plasticizers include: triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness should be sufficient to ensure that the oral dosage form remains intact until the desired site of topical delivery in the lower intestinal tract is reached. Colorants, detackifiers, surfactants, antifoaming agents, lubricants, stabilizers such as hydroxypropylcellulose, acid/base may be added to the coatings besides plasticizers to solubilize or disperse the coating material, and to improve coating performance and the coated product.

An exemplary methacrylic copolymer is Eudragit L®, particularly L30D® and Eudragit 100-55®, manufactured by Rohm Pharma, Germany. In Eudragit L-30 D®, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another methacrylic acid polymer is Eudragit S®, manufactured by Rohm Pharma, Germany. Eudragit S differs from Eudragit L-30-D only insofar as the ratio of free carboxyl groups to ester groups is approximately 1:2. Eudragit S is insoluble at pH below 5.5, but unlike Eudragit L-30-D, is poorly soluble in gastrointestinal fluids having pH of 5.5-7.0, such as is present in the small intestine media. This copolymer is soluble at pH 7.0 and above, i.e., the pH generally found in the colon. Eudragit S can be used alone as a coating to provide delivery of beginning at the large intestine via a delayed release mechanism. In addition, Eudragit S, being poorly soluble in intestinal fluids below pH 7, can be used in combination with Eudragit L-30-D, soluble in intestinal fluids above pH 5.5, in order to effect a delayed release composition. The more Eudragit L-30 D used the more proximal release and delivery begins, and the more Eudragit S used, the more distal release and delivery begins. Both Eudragit L-30-D and Eudragit S can be substituted with other pharmaceutically acceptable polymers with similar pH solubility characteristics.

A coating process frequently involves spraying a coating solution onto a substrate. The coating solution can be a molten solution of the encapsulation coat composition free of a dispersing medium. The coating solution can also be prepared by solubilizing or suspending the composition of the encapsulation coat in an aqueous medium, an organic solvent, a supercritical fluid, or a mixture thereof. At the end of the coating process, the residual dispersing medium can be further removed to a desirable level utilizing appropriate drying processes, such as vacuum evaporation, heating, freeze drying, etc.

A pelletization process typically involves preparing a molten solution of the composition of the solid carrier or a dispersion of the composition of the solid carrier solubilized or suspended in an aqueous medium, an organic solvent, a supercritical fluid, or a mixture thereof. Such solution or dispersion is then passed through a certain opening to achieve the desired shape, size, and other properties. Similarly, appropriate drying processes can be adopted to control the level of the residual dispersing medium, if necessary.

Surfactants can be used in formulating coated bead compositions to provide a wetting function, to enable hydrophobic drugs to properly adhere to beads and/or water-soluble binders. For example, U.S. Pat. No. 4,717,569 discloses coated bead compositions of hydrophobic steroid compounds wetted by a hydrophilic surfactant and adhered to the beads by a water-soluble binder. The steroid compound is present as finely divided particles, held to the beads by the binder. Surfactants at higher levels, i.e., in amounts far in excess of the amounts necessary or appropriate for a wetting function, enable a pharmaceutical active ingredient to be fully or at least partially solubilized in the encapsulation coating material itself, rather than merely physically bound in a binder matrix. Binders may be unnecessary. The amount of hydrophilic surfactant can be adjusted so as to at least partially or fully solubilize the pharmaceutical active ingredient, with the optional lipophilic surfactants, triglycerides and solubilizer chosen to further increase the pharmaceutical active ingredient's solubility. The encapsulation coat can alternatively be formulated without the active ingredient. An active ingredient can be provided in the composition itself but not in the encapsulation coat. Such a formulation delivers the active ingredient to the patient along with the surfactants or other components to facilitate dispersion (emulsification/micellization). The optional lipophilic surfactant and triglycerides can be used as desired to further enhance solubilization of the ebselen or ebsulfur derivatives, or to promote dispersion (emulsification/micellization) in vivo, or to promote in vivo absorption at the absorption site.

Other known delayed release and solubilization technologies may be employed.

Methods of Treatment

The formulation may be provided to a patient or subject in need of such treatment for at least one of: (1) preventing the disease; for example, preventing a disease, condition or disorder in an individual who may be predisposed to the disease, condition or disorder but does not yet experience or display the pathology or symptoms of the disease; or decreasing the likelihood of a relapse of a disease, condition, or disorder in an individual; (2) inhibiting the disease; for example, inhibiting a disease, condition or disorder in an individual who is experiencing or displaying the pathology or symptoms of the disease, condition or disorder (i.e., arresting further development of the pathology and/or symptoms) such as lowering the bacterial load in the case of a bacterial infection, and (3) ameliorating the disease; for example, ameliorating a disease, condition or disorder in an individual who is experiencing or displaying the pathology or symptoms of the disease, condition or disorder (i.e., reversing the pathology and/or symptoms) such as reducing infection-related tissue damage in the case of a bacterial infection.

In the case of a chronic disease associated with inflammatory cascade activation, the goal of therapy is to damp the inflammatory responses, and to the extent possible, reduce the continuing trigger for inflammation. For example, in SLE, it is believe that a trigger for inflammation is the defective clearance of apoptotic cells, which result in anti-nuclear antibodies, etc., which then result in autoimmune responses. Glutathione and ebselen (or the other effective derivatives as encompassed herein) facilitate normalization of cellular responses. Note that apoptosis is associated with loss of GSH in the apoptotic cell, and therefore the mere administration of GSH has potentially contradictory effects on facilitating efficient apoptosis. Likewise, high levels of GSH interrupt at least some of the anti-inflammatory effects of ebselen, e.g., inhibition of lipooexygenase.

In some embodiments, the ebselen or ebsulfur derivative medication is administered at a dose of from 1 mg to four grams (e.g., from 25 mg to three grams, from 100 mg to two grams, from one to two grams). In some embodiments, the ebselen derivative medication is administered at a dose of at least 40 mg (e.g., at least 100 mg, at least 500 mg, at least one gram, at least two grams, or at least three grams) and/or at most four grams (e.g., at most three grams, at most two grams, at most one gram, at most 500 mg, or at most 100 mg). Preferably, the maximum amount of ebselen or ebsulfur derivative per capsule is 250 mg. and larger doses are provided in multiple capsules. The glutathione component may be provided in an amount of 250-1000 mg. per capsule, with a charge complex neutralizing agent, e.g., reduced ascorbic acid flake crystals, provided in about >2× molar excess. For example, a capsule may contain 500 mg reduced L-glutathione, 250 mg ascorbic acid, and 50 mg ebselen, enteric-release coated granules, per capsule. Capsules are taken two at a time, four times a day, on an empty stomach.

In some embodiments, the combination can be administered to a subject (e.g., a human subject) at a frequency of at least one dose per day (e.g., at least one dose per 12 hours, or at least one dose per 6 hours) and/or at most one dose per three hours (e.g., at most one dose per six hours, or at most one dose per 12 hours). In some embodiments, the ebselen or ebsulfur derivative medication are administered to a subject (e.g., a human subject) at a frequency of from one dose per day to one dose per three hours (e.g., from one dose per day to one dose per six hours, from one dose per day to one dose per 12 hours).

In some embodiments, a treatment cycle can last at least one day, though in a preferred embodiment, the intended use is to treat a chronic disease with an inflammatory component, such as diabetes or systemic lupus erythematosus. In such conditions, there may be a concurrent microbial infection, which may be treated with antibiotics, if the infection does not respond to the ebselen or ebsulfur derivative itself.

The formulation can be administered to a subject (e.g., a human subject) at a dosage of at least one dose per day (e.g., at least one dose per 12 hours, or at least one dose per 6 hours) and/or at most one dose per three hours (e.g., at most one dose per six hours, or at most one dose per 12 hours). In some embodiments, the formulation is administered to a subject (e.g., a human subject) at a dosage of from one dose per day to one dose per three hours (e.g., from one dose per day to one dose per six hours, from one dose per day to one dose per 12 hours).

In some embodiments, the ebselen or ebsulfur derivative component of the formulation is a controlled-release composition, e.g., designed to selectively release a therapeutic agent in a desired area of the small and/or large intestines, and/or gradually release the therapeutic agent over a selected area of the small and/or large intestines. The controlled-release composition can include articles (e.g., beads, tablets, pills, capsules) including a therapeutic agent. The articles can be coated with a controlled-release coating. The controlled-release coating provides a protective barrier for the therapeutic agent against acidic environments (e.g., the stomach) so that the formulation passes through the stomach with little (e.g., no) therapeutic agent being released, and so that the therapeutic agent is relatively easily released in less acidic environments (e.g., the intestines, the colon). In some embodiments, the controlled-release coating can control the release the therapeutic agent in a desired area of the small and/or large intestines, and/or gradually release the therapeutic agent over a selected area of the small and/or large intestines.

In some embodiments, the controlled-release composition includes a controlled-release bead (e.g., a bead having controlled-release properties and/or a controlled-release coating, an enteric-coated bead), which may have a variety of cross-sectional shapes, such as a circle, an ellipse, a regular polygon (e.g., a square, a diamond, a pentagon, a hexagon, or an octagon), and/or an irregular polygon. For example, in some embodiments, the bead is a sphere and has a circular cross-section. The bead can have a maximum average dimension (e.g., a diameter) of from 0.1 to three mm (e.g., from 0.2 to two mm, from 0.4 to one mm, or from one to two mm). In some embodiments, the bead can have a maximum average dimension of at least 0.1 mm (e.g., at least 0.5 mm, at least one mm, at least 1.5 mm, at least two mm) and/or at most three mm (e.g., at most two mm, at most 1.5 mm, at most one mm, or at most 0.5 mm). The maximum average dimension of a bead is determined by measuring the maximum dimension of each bead in a population of beads, adding the maximum dimension of each bead, and dividing the sum by the number of measured beads. In some embodiments, the controlled-release bead has a core that includes a biocompatible and/or bioabsorbable material such as a carbohydrate (e.g., sugar, starch, sodium carboxymethylcellulose, cellulose, alginates, and/or sodium starch glycolate). The controlled-release bead can have a surface covered with a controlled-release coating (e.g., an enteric coating). The coating can include a material that is stable in acidic environments, but that disintegrates relatively rapidly in less acidic environment. Examples of controlled-release coatings include beeswax, beeswax and glyceryl monostearate, shellac and cellulose, cetyl alcohol, mastic and shellac, shellac and stearic acid, polyvinyl acetate and ethyl cellulose, neutral copolymer of polymethacrylic acid ester (Eudragit L30D), copolymer of methacrylic acid and methacrylic acid methylester (Eudragit S), neutral copolymers of polymethacrylic acid esters containing metallic stearates, neutralized hydroxypropyl methylcellulose phthalate polymer, and/or combinations thereof.

In some embodiments, the controlled-release composition can include more than one type of controlled-release beads, each type having any combination of therapeutic agent, maximum average dimension, concentration, distribution of therapeutic agent, core materials, and/or coating materials.

In some embodiments, the controlled-release beads are made by extruding beads of a granulated wet mixture of core materials (e.g., a carbohydrate), a binder and/or a therapeutic agent, and by placing the extrudate into a spheronizer. In some embodiments, a bead having a core without any therapeutic agents can be sprayed with a solution and/or a dispersion (e.g., a nanodispersion) of a therapeutic agent. The weight percent of the therapeutic agent can be determined by measuring the bead before and after coating, or by pre-measuring the mass of each component of a bead prior to forming a mixture of core materials. Methods for making coated compositions are described, for example, in U.S. Pat. Nos. 7,217,429, and 6,224,910. In some embodiments, the bead core are commercially available (e.g., from Chr. Hansen, Denmark).

In some embodiments, the controlled-release beads are pressed into a tablet or a pill, encapsulated in a capsule, or suspended in a solution to form a suspension. In some embodiments, a tablet, pill, or capsule can be formed directly from a therapeutic agent and any of a number of excipients, binders, and/or fillers. The tablet, pill, or capsule can contain varying percentage amounts of the therapeutic agents and carriers. For example, the tablet, pill, or capsule can contain more than 0.01 percent (e.g., more than 0.1 percent, more than one percent, more than five percent, or more than 10 percent) and/or less than 20 percent by weight (e.g., less than 10 percent, less than five percent, less than one percent, or less than 0.1 percent) of the therapeutic agent (e.g., a metal-containing material and/or a non-metal antibiotic medication). Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The tablet, pill, or capsule can then be coated with a controlled-release coating.

Examples of formulations and delivery methods to the intestinal tract for increased absorption are described, for example, in Davis, Drug Discovery Today, 10(4) 2005, 249-257; Fell, J. Anat. (1996) 189, 517-519; Ibekwe et al., The Drug Discovery Companies Report Spring/Summer 2004 (2004) 27-30.

The formulation can be used to treat, for example a human or an animal (e.g., a dog, a cat, a horse, a bird, a reptile, an amphibian, a fish, a turtle, a guinea pig, a hamster, a rodent, a cow, a pig, a goat, a primate, a monkey, a chicken, a turkey, a buffalo, an ostrich, a sheep, a llama).

The ebselen derivatives, dosage form, or compositions, can be administered by a number of routes, including but not limited to orally, intravenously, intramuscularly, intraperitoneally, intranasally, as an inhaled powder, rectally, vaginally, buccaly, transdermally, or parenterally, in form of solid, semi-solid, micronized powder, lyophilized powder, or liquid. For example, the composition can be used in the form of tablet, suppository, pill, soft and hard gelatin capsule, granule, solution, suspension or aerosol. Preferred is single unit form for exact dosage. The pharmaceutical composition includes conventional excipient or carrier and one or more ebselen or ebsulfur derivatives. In some cases, a prodrug form may be provided, which is converted into an active benzisoselenazolonyl or benzisothiazolonyl derivative after administration.

The ebselen or benzisoselenazolonyl or ebsulfur or benzisothiazolonyl derivatives may be administered together with, before or after the glutathione. Antimicrobial agents may also be employed concurrently, before or after. One or more ebselen or ebsulfur derivatives may be administered through the same route of administration or a different route of administration as the glutathione.

Doses

In another aspect, a composition, dosage form, or an active agent disclosed herein can be present or administered in at least about 1 mg, for example, at least about: 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, or 1 g; or about 1 to about 500 mg, for example, about 1-50 mg, about 1-25 mg, about 1-20 mg, about 1-15 mg, about 1-10 mg, about 10-20 mg, about 10-50 mg, about 10-100 mg, about 10-150 mg, about 20-25 mg, about 20-50 mg, about 20-100 mg, about 20-150 mg, about 20-200 mg, about 20-250 mg, about 50-250 mg, about 50-200 mg, about 50-150 mg, about 50-100 mg, about 100-150 mg, about 100-200 mg, about 100-300 mg, about 100-500 mg, about 150-200 mg, about 150-250 mg, about 200-500 mg, or about 250-500 mg.

Pharmacokinetics

The pharmacokinetic data disclosed herein (e.g., Cₘₐₓ, Tₘₐₓ, AUC₀₋₁₅ₘᵢₙ, AUC₀₋₃₀ₘᵢₙ, AUC_{0-inf}, T_{1/2}) can be measured from a primate, for example a human, after a composition disclosed herein is administered. The composition comprising glutathione and the isoselenazol or isothiazol derivative does not degrade a pharmacokinetic parameter of the glutathione by more than about 15%, when compared to administration of glutathione in corresponding single drug therapy dosage form alone (i.e., without the isoselenazol or isothiazol derivative), as measured by a same method. In some embodiments, the dosage form might result in an improvement of a pharmacokinetic parameters, and in such cases improvement may be 20%, 25%, 30%, 35%, 40%, 45%, 50%, 55%, 60%, 65%, 70%, 75%, 80%, 85%, 90%, 95%, 100%, 150%, 200%, or greater. In some embodiments, the improvement may be 15% to 50%, 15% to 25%, or 25% to 50%, 25% to 75%, 50% to 75%, 50% to 100%, 100% to 150%, or 100% to 200%.

In some embodiments, the methods and compositions disclosed herein comprise a mean Tₘₐₓ of the isoselenazol or isothiazol derivative after administration of the composition of at least about 1 minutes, for example, at least about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 25 minutes, 30 minutes, 35 minutes, 40 minutes, 45 minutes, 50 minutes, 60 minutes, 90 minutes, or 120 minutes. The mean Tₘₐₓ of an the isoselenazol or isothiazol derivative after administration of the composition can be about 1 to about 120 minutes, for example, about 1-120 minutes, about 1-90 minutes, about 1-60 minutes, about 1-50 minutes, 1-40 minutes, 1-30 minutes, 1-20 minutes, 1-10 minutes, 1-5 minutes, about 1-2 minutes, about 5-120 minutes, about 5-90 minutes, about 5-60 minutes, about 5-50 minutes, 5-40 minutes, 5-30 minutes, 5-25 minutes, 5-20 minutes, 5-10 minutes, about 10-120 minutes, about 10-90 minutes, about 10-60 minutes, about 10-50 minutes, 10-40 minutes, 10-30 minutes, 10-20 minutes, about 20-120 minutes, about 20-90 minutes, about 20-60 minutes, about 20-50 minutes, 20-40 minutes, 20-30 minutes, about 30-120 minutes, about 30-90 minutes, about 30-60 minutes, about 30-50 minutes, 30-40 minutes, about 40-120 minutes, about 40-90 minutes, about 40-60 minutes, 40-50 minutes, about 50-120 minutes, about 50-90 minutes, about 50-60 minutes, about 60-120 minutes, about 60-90 minutes, or about 90-120 minutes.

In some embodiments, the methods and compositions disclosed herein comprise a mean Cₘₐₓ of the isoselenazol or isothiazol derivative (expressed as weight of administered drug component before metabolic alteration) after administration of the composition of at least about 0.1 nanogram/milliliter (ng/mL), for example, at least about 0.1 ng/mL, 0.2 ng/mL, 0.3 ng/mL, 0.4 ng/mL, 0.5 ng/mL, 0.6 ng/mL, 0.7 ng/mL, 0.8 ng/mL, 0.9 ng/mL, 1 ng/mL, 1.5 ng/mL, 2 ng/mL, 2.5 ng/mL, 3 ng/mL, 3.5 ng/mL, 4 ng/mL, 4.5 ng/mL, 5 ng/mL, 5.5 ng/mL, 6 ng/mL, 6.5 ng/mL, 7 ng/mL, 7.5 ng/mL, 8 ng/mL, 8.5 ng/mL, 9 ng/mL, 9.5 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, 225 ng/mL, 250 ng/mL, 275 ng/mL, 300 ng/mL, 333 ng/mL, 367 ng/mL, 400 ng/mL, 450 ng/mL, 500 ng/mL, 600 ng/mL, 700 ng/mL, 800 ng/mL, 900 ng/mL, 1000 ng/mL, 1250 ng/mL, 1500 ng/mL, 1750 ng/mL, 2000 ng/mL, 2500 ng/mL, or 3000 ng/mL. The mean Cₘₐₓ of the isoselenazol or isothiazol derivative after administration of the composition can be about 0.1 to about 3000 ng/mL, for example, about 0.1-3000 ng/mL, 1-2000 ng/mL, 5-1000 ng/mL, 5-500 ng/mL, 10-250 ng/mL, 10-200 ng/mL, 15-200 ng/mL, 20-150 ng/mL, 20-125 ng/mL, 20-100 ng/mL, 10-100 ng/mL, 5-150 ng/mL, 5-130 ng/mL, 5-110 ng/mL, 5-100 ng/mL, 5-90 ng/mL, 5-75 ng/mL, 5-30 ng/mL, 1-10 ng/mL, 1-5 ng/mL, 5-150 ng/mL, 5-130 ng/mL, 5-110 ng/mL, 5-90 ng/mL, 5-70 ng/mL, 5-50 ng/mL, 5-30 ng/mL, 5-10 ng/mL, 10-150 ng/mL, 10-130 ng/mL, 10-110 ng/mL, 10-90 ng/mL, 10-70 ng/mL, 10-50 ng/mL, 10-30 ng/mL, 30-150 ng/mL, 30-130 ng/mL, 30-110 ng/mL, 30-90 ng/mL, 30-70 ng/mL, 30-50 ng/mL, 50-150 ng/mL, 50-130 ng/mL, 50-110 ng/mL, 50-90 ng/mL, 50-70 ng/mL, 70-150 ng/mL, 70-130 ng/mL, 70-110 ng/mL, 70-90 ng/mL, 90-150 ng/mL, 90-130 ng/mL, 2.5-100 ng/mL, 75-125 ng/mL, 100-150 ng/mL, or 75-150 ng/mL.

In some embodiments, the methods and compositions disclosed herein comprise a mean T_{1/2} of the isoselenazol or isothiazol derivative after administration of the composition of at least about 20 minutes, for example, at least about 20 minutes, 30 minutes, 40 minutes, 50 minutes, 60 minutes, 70 minutes, 80 minutes, 90 minutes, 100 minutes, 120 minutes, 150 minutes, 200 minutes, 250 minutes, or 300 minutes. The mean T_{1/2} of an active agent after administration of the composition can be about 20 to about 600 minutes, for example, about 20-600 minutes, 20-300 minutes, 30-200 minutes, 30-150 minutes, 30-120 minutes, 45-100 minutes, 45-90 minutes, 30-60 minutes, 20-45 minutes, 20-300 minutes, 20-250 minutes, 20-200 minutes, 20-150 minutes, 20-120 minutes, 20-100 minutes, 20-80 minutes, 20-60 minutes, 20-40 minutes, 40-300 minutes, 40-250 minutes, 40-200 minutes, 40-150 minutes, 40-120 minutes, 40-100 minutes, 40-80 minutes, 40-60 minutes, 60-300 minutes, 60-250 minutes, 60-200 minutes, 60-150 minutes, 60-120 minutes, 60-100 minutes, 60-80 minutes, 80-300 minutes, 80-250 minutes, 80-200 minutes, 80-150 minutes, 80-120 minutes, 80-100 minutes, 100-300 minutes, 100-250 minutes, 100-200 minutes, 100-150 minutes, 100-120 minutes, 120-300 minutes, 120-250 minutes, 120-200 minutes, 120-150 minutes, 150-300 minutes, 150-250 minutes, 150-200 minutes, 200-300 minutes, 200-250 minutes, or 250-300 minutes.

It is therefore an object to provide a method of treating a mammal having a chronic inflammatory disorder, comprising coadministering glutathione and an isoselenazol or isothiazol derivative, each in an effective amount, and according to an efficacious regimen, to treat the chronic inflammatory disorder.

It is a further object to provide a pharmaceutically acceptable unit dosage form for treating a chronic inflammatory condition of a mammal, comprising at least one isoselenazol or isothiazol derivative, and glutathione, each provided in an efficacious amount to treat the chronic inflammatory condition.

It is a further object to provide a pharmaceutically acceptable unit dosage form for treatment of a chronic inflammatory disorder, comprising at least 500 mg reduced L-glutathione, at least 100 mg ascorbic acid, and at least 25 mg of an isoselenazol or isothiazol derivative which is a mammalian glutathione peroxidase mimic, and a bacterial thioredoxin reductase inhibitor. The unit dosage form may have an immediate release portion comprising glutathione and ascorbic acid, and a delayed release portion comprising the isoselenazol or isothiazol derivative, wherein the glutathione is physically separated within the unit dose form from the isoselenazol or isothiazol derivative.

The glutathione and the isoselenazol or isothiazol derivative may be provided together within a bioavailable dosage form. The glutathione and the isoselenazol or isothiazol derivative may be chemically separated within the dosage form, the isoselenazol or isothiazol derivative is provided within a delayed release formulation, and the glutathione provided within an immediate release formulation.

The isoselenazol or isothiazol may comprise at least one compound according to Formula I or a pharmaceutically acceptable salt or derivative thereof:

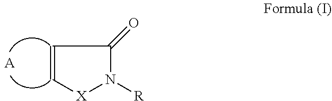

wherein A represents a saturated, unsaturated or polyunsaturated 3 to 6 member carbon chain and wherein N may optionally substitute for one or more carbons, and which is optionally substituted with one or more of OR, SR, and alkylamino, C₁-C₅ alkyl, OH, alkoxyl, SH, NH₂, N-alkylamino, N,N-dialkylamino, COOH, CHO, NO₂, F, Cl, Br, or I; wherein X is selenium or sulfur, and wherein R is selected from the group consisting of: (i) H; (ii) alkyl having a carbon chain of 1 to 14 carbon atoms wherein the carbon chain is branched or unbranched which is optionally substituted with bensisoselenazol-3(2H)-one-2-yl, bensisotiazol-3(2H)-one-2-yl, OH, alkoxyl, SH, NH₂, N-alkylamino, N,N-dialkylamino, COOH, aryl which is optionally substituted with C₁-C₅ alkyl, OH, alkoxyl, SH, NH₂, N-alkylamino, N,N-dialkylamino, COOH, CHO, NO₂, F, Cl, Br, I, or heteroaryl which is optionally substituted with C₁-C₅ alkyl, OH, alkoxyl, SH, NH₂, N-alkylamino, N,N-dialkylamino, COOH, CHO, NO₂, F, Cl, Br, and I; (iii) aryl which is optionally substituted with C₁-C₅ alkyl, OH, alkoxyl, SH, NH₂, N-alkylamino, N,N-dialkylamino, COOH, CHO, NO₂, F, Cl, Br, or I; and (iv) heteroaryl which is optionally substituted with C₁-C₅ alkyl, OH, alkoxyl, SH, NH₂, N-alkylamino, N,N-dialkylamino, COOH, CHO, NO₂, F, Cl, Br, or I. Ebselen (X=Se) and Ebsulfur-23(X=S) are preferred compositions. The chronic inflammatory disorder may comprise systemic lupus erythematosus or diabetes mellitus Type II, for example.

The glutathione is preferably reduced L-glutathione, pharmaceutically stabilized with a molar excess amount of ascorbic acid. The ascorbic acid may be provided in a flake crystal form, the reduced L-glutathione is provided in a powder form, and the ascorbic acid flake crystals form a dense charge transfer complex with the reduced L-glutathione powder. The dosage form may be substantially devoid of oxidant compositions. The dosage form may be packed in a single dose pack under an inert gas. The dosage form may be packed in a multidose pack under an inert gas. The dosage form may be packed in an oxygen barrier package containing an oxygen absorbing insert. The glutathione may be present in an amount of between about 250-2000 mg per dosage form. The isoselenazol or isothiazol derivative may be present in an amount of between about 1-250 mg per dosage form. Ascorbic acid may be is provided in an amount of at least 100 mg per dosage form.

The dosage form may further comprise a pharmaceutically acceptable antibiotic.

The glutathione and isoselenazol or isothiazol derivative may be together provided as a pharmaceutically acceptable dosage form in an integral capsule comprising reduced L-glutathione and a relative molar excess of ascorbic acid in a charge transfer complex, which is released immediately after administration in a stomach of the mammal, and at least one delayed release granule comprising the isoselenazol or isothiazol derivative, such that during storage prior to administration the glutathione and isoselenazol or isothiazol derivative are physically isolated, and the isoselenazol or isothiazol derivative is not released from the at least one delayed release granule until after the glutathione is absorbed.

The isoselenazol or isothiazol derivative may be provided within a delayed release portion which is physically isolated from the glutathione within a common unit dosage form. The delayed release form may comprise an outer coating which dissolves after passage through the stomach, and a surfactant to facilitate dissolution of the isoselenazol or isothiazol derivative. The isoselenazol or isothiazol derivative may be dispersed within a slowly dissolving matrix.

### DETAILED DESCRIPTION OF THE PREFERRED EMBODIMENTS

Compounds Synthesis:

Benzisoselenazol-3(2H)-one (1-11) and bisbenzisoselenazol-3(2H)-one (12-14) derivatives were synthesized by the treatment of 2-(chloroseleno)benzoyl chloride, which was obtained from anthranilic acid [30], with corresponding amines or diamines using the reported procedure with minor modifications (Scheme 1). Disodium diselenide used in this procedure was prepared by the reaction of sodium borohydride with selenium in water, instead of the reaction between sodium and selenium in THF. This method is safer, as the unreacted sodium, if there is any, may cause an explosion in the next step of the reaction, which has to be carried out in water. Synthesis of 7-azabenzisoselenazol-3(2H)-ones were reported. However, when the synthesis of 2-(5-chloro-2-pyridyl)-7-azabenzisoselenazol-3(2H)-one (17) was repeated, and formation of different organoselenium compounds were observed depending on amount of thionyl chloride, nature of solvent and reaction time. Formation of selenamide (20) and diethyl 2, 2′-diselenobisnicotinate (21) was observed when dichloromethane used as received to extract the 2-(chloroseleno)nicotinoyl chloride (16) and for further cyclization with 5-chloro-2-aminopyridine. The products were easily separated by column chromatography using dichloromethane as eluent. This reaction was reproducible. Expected product 17 was obtained as major product along with bis(2-carbamoyl)phenyl diselenide (18) when the dry dichloromethane or acetonitrile used as solvent and refluxed for 36 hrs. When the reflux time is reduced to 16 hr or less with same amount of thionyl chloride compound 18 was obtained as major product. The reactions are shown in Scheme 2. Formation of 20 may be explained via the formation ethyl 2-(chloroseleno)nicotinate (19) due to the reaction of ethyl alcohol present in dichloromethane as impurity with the hard electrophilic center of chloride (16) localized on the carbonyl carbon atom. o-Acylation is expected to proceed faster than o-selenenylation as reported. Acyclic selenamides are very unstable and so far only few reports about acyclic selenamides are known in the literature. The stability of selenamide (20) may be due to the presence of Se . . . O intramolecular interaction between the carbonyl oxygen of carboxyl ethyl group and selenium. The stabilization organoselenium compounds by such type of Se . . . O intramolecular interactions have been extensively studied in the literature. All reactions were performed under inert atmosphere using Schlenk techniques. All solvents were purified by the standard procedures^{[37]} and were freshly distilled prior to use. All chemicals were purchased from Sigma-Aldrich or Lancaster and used as received. ¹H NMR spectra were recorded in CDCl₃ or DMSO-d₆ on a Varian VXR spectrometer operating at 400 MHz and chemical shifts are reported in ppm relative to TMS. Benzisoselenazol-3(2H)-one (1-11) and bisbenzisoselenazol-3(2H)-one (12-14) were prepared from 2-(chloroseleno)benzoyl chloride using the synthetic procedure described in the literature with slight modifications. Diselenide of nicotinic acid (17) were also synthesized by reported method.

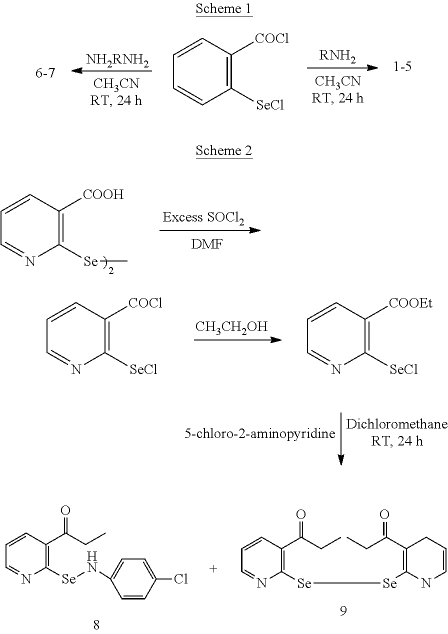

### Synthesis of 2-(5-chloro-2-pyridyl)-7-azabenzisoselenazol-3(2H)-one (17)

1 g of 15 is suspended in 50 ml of thionyl chloride and one drop of dimethylformamide was added, and the reaction mixture was refluxed for 36 hr. The excess thionyl chloride was removed under reduced vacuum and dichloride 16 was occurred as yellow crystalline solid. Due to its low stability dichloride 16 (2 mmole) was dissolved in 50 ml dry dichloromethane or acetonitrile and the solution of 5-chloro-2-aminopyridine (6 mmole) dissolved in dichloromethane or acetonitrile was added dropwise at ice/salt temperature. After 24 hr the solvent was evaporated. The residue was purified by column chromatography using dichloromethane as eluent. Yield (40%). ¹H NMR (400 MHz, DMSO-d₆, ppm): 67.54 (dd, 1H), 8.04 (dd, 1H), 8.24 (d, 1H), 8.50 (s, 1H), 8.61 (d, 1H), 8.87 (d, 1H).

### Synthesis of Ethyl 2-(5-chloro-2-pyridylamidoseleno)nicotinate (20) and diethyl 2, 2′-diselenobisnicotinate (21)

A suspension of 15 (1 g) in thionyl chloride (7 ml) and one drop of dimethylformamide were refluxed for 8 h. After this period further 7 ml of thionyl chloride and one drop of dimethyl formamide was added and refluxed for further 12 h. The excess thionyl chloride was evaporated under reduced pressure and the residue was dissolved in dichloromethane and filtered under inert conditions. From the filtrate the dichloromethane was evaporated to obtain the dichloride (19) as yellow crystalline solid. To the ice/salt bath solution of dichloride (2.5 mmole) dissolved in dichloromethane was added dropwise the solution of 5-chloro-2-aminopyridine (7.5 mmole), and the reaction was continued for 3 h. After this period the reaction mixture was washed with water thrice (3×20 ml) and the organic layer was separated and dried in anhydrous sodium sulfate. The solvent was removed under reduced pressure and product was further purified by column chromatography using dichloromethane as eluent to get 20 and 21. Compound 20 is white crystalline substance and compound 21 is pale yellow color. Compound 20: Yield, 35%. ¹H NMR (400 MHZ, DMSO-d₆, ppm): δ 1.38 (t, CH₃), 4.40 (q, CH₂), 6.85 (d, ArH), 7.35 (dd, ArH), 7.5 (dd, ArH), 7.98 (s, NH), 8.02 (s, ArH), 8.25 (dd, ArH), 8.6 (dd, ArH). Compound 21: Yield, 20%. ¹H NMR (400 MHz, CDCl₃, ppm): 61.4 (t, CH₃), 4.4 (q, CH₂), 7.10 (dd ArH), 8.2 (dd, ArH), 8.45 (dd, ArH)

Glutathione Peroxidase (GPx) Activity Assay:

GPx activity of ebselen derivatives was performed in the potassium phosphate buffer, pH 7.4 containing 240 μM NADPH, 0.5 mM GSH, and 0.5 unit of glutathione reductase with 30 μM compound in the presence of 0.5 mM of H₂O₂. The absorbance at 340 nm was followed for 10 min, and GPx activity was calculated in terms of NADPH consumption per minute. The sample in the absence of H₂O₂ was used as the control.

Measurement of IC50 of Ebselen Derivatives for Mammalian TrxR:

The inhibition of ebselen derivatives on mammalian TrxR was performed in the 50 mM Tris-HCl, pH 7.5 buffer containing 100 nM recombinant rat TrxR, 200 μM NADPH. The compounds of different concentration (0.02-10 μM) were incubated for 10 minute and then 1 mM DTNB was added to assay TrxR activity by following the initial linear increase at A412 for 2 minutes. The sample incubated with DMSO was used as the control.

Cell Viability Experiment:

Human embryonic kidney cells (HEK 293T) were cultured in RPMI 1640 (GIBCO) supplemented with 2 mM L-glutamine, 10% FCS, 100 units/ml penicillin, and 100 μg/ml streptomycin at 37° C. in a 5% CO₂ incubator. HEK 293T cells were plated at a density of 1×10⁴ cells/well in 96 micro-well plates and allowed to grow in the growth medium for 24 h. Then different concentrations of ebselen derivatives were added in the medium, and incubation was conducted for another 24 h. Cell proliferation and viability were determined using an XTT kit (Roche). After XTT agents were added, the cells were grown for another 3 hours. The data are the means of three experiments and at least repeated twice.

### Example 1

Reduced L-glutathione, a naturally-occurring water-soluble tripeptide (gamma-glutamyl-cysteinyl-glycine) is the most prevalent intracellular thiol in most biological systems. A preferred formulation of glutathione according to the present invention provides capsules for oral use containing 500 mg reduced L-glutathione, 250 mg USP grade crystalline ascorbic acid, and not more than 0.9 mg magnesium stearate, NF grade in an OO-type gelatin capsule.

Also within the capsule is provided enteric release ebselen, 100-500 mg, as granules of ebselin in a binder/solubilizer coated with an enteric release film. The formulation may administered 1-4 capsules by mouth, two to four times daily, preferably on a empty stomach. For example, a capsule may contain 100, 200, 250, 300, 330, 400 or 500 mg of ebselin or another isoselenazol or isothiazol derivative which is effective as a mammalian glutathione peroxidase mimic. The granules preferably are designed to release the ebselen or other isoselenazol or isothiazol derivative after the ligament of Treitz, and for example, may have a solubility that is insoluble in acid and increases in solibility neutral or basic solutions, i.e., in the presence of pacrteatic secretions, or includes a shell which is hydrolyzed by bile acids or pancreatic enzymes.

As noted in the literature, ebselen doses in the range of 10-30 mg/kg i.p.or i.v or p.o. have been found particularly effective for treatment of diseases. Lindenblatt, Nicole, et al. “Anti-oxidant ebselen delays microvascular thrombus formation in the rat cremaster muscle by inhibiting platelet P-selectin expression.” Thrombosis and haemostasis 90.5 (2003): 882-892; Lapchak, Paul A., and Justin A. Zivin. “Ebselen, a seleno-organic antioxidant, is neuroprotective after embolic strokes in rabbits synergism with low-dose tissue plasminogen activator.” Stroke 34.8 (2003): 2013-2018; Dawson, D. A., et al. “The neuroprotective efficacy of ebselen (a glutathione peroxidase mimic) on brain damage induced by transient focal cerebral ischaemia in the rat.” Neuroscience letters 185.1 (1995): 65-69.

The capsule is preferably a standard two-part hard gelatin capsule of double-0 (00) size, which may be obtained from a number of sources. After filling, the capsules are preferably stored under nitrogen, to reduce oxidation during storage. The capsules are preferably filled according to the method of U.S. Pat. No. 5,204,114, using crystalline ascorbic acid as both an antistatic agent and stabilizer. Further, each capsule preferably contains 500 mg of glutathione and 250 mg of crystalline ascorbic acid. A preferred composition includes no other excipients or fillers; however, other compatible fillers or excipients may be added. While differing amounts and ratios of glutathione and stabilizer may be used, these amounts are preferable because they fill a standard double-O capsule, and provide an effective stabilization and high dose. Further, the addition of calcium carbonate, a component of known high dose glutathione capsules, is avoided as there may be impurities in this component. Further, calcium carbonate acts as a base, neutralizing stomach acid, which accelerates degradation of glutathione in the small intestine.

Because the glutathione and ascorbic acid are administered in relatively high doses, it is preferred that these components be highly purified, to eliminate impurities, toxins or other chemicals, which may destabilize the formulation or produce toxic effects or side effects. As stated above, the formulation may also include other pharmaceutical agents, of various classes.

### Example 2

The preferred regimen for treatment of humans with glutathione according to the present invention is the administration of between 1 and 10 grams per day, in two divided doses, between meals (on an empty stomach), of encapsulated, stabilized glutathione and ebselen according to Example 1.

### Example 3

A formulation disclosed herein is administered to a human at a dosage disclosed herein, which results in a pharmacokinetic parameter disclosed herein.

The foregoing disclosure of embodiments and exemplary applications of the present invention has been presented for purposes of illustration and description. It is not intended to be exhaustive or to limit the invention to the precise forms disclosed.

What is claimed is:

1. A method, comprising coadminstering to a mammal:
(a) a glutathione component, comprising glutathione, or a pharmaceutically acceptable salt thereof; and
(b) an azol component, comprising at least one of:
an isoselenazol,
an isoselenazol derivative,
an isothiazol,
an isothiazol derivative, and
a pharmaceutically acceptable salt of the isoelenazol, the isoselenazol derivative, the isothazol, or the isothiazol derivative,
each of the glutathione component and the azol component being present in a therapeutically effective amount.

2. The method according to claim 1, wherein the mammal is a human having a chronic inflammatory disorder, and the glutathione component and the azol component are each individually present in a therapeutically effective amount to treat the chronic inflammatory disorder or to ameliorate a symptom associated with the chronic inflammatory disorder.

3. The method according to claim 2, wherein the chronic inflammatory disorder is selectively associated with one or more of the group consisting of systemic lupus erythematosus, diabetes mellitus type II, acne vulgaris, asthma, an autoimmune disease, an autoinflammatory disease, a celiac disease, chronic prostatitis, diverticulitis, glomerulonephritis, hidradenitis suppurativa, a hypersensitivity, an inflammatory bowel disease, interstitial cystitis, otitis, a pelvic inflammatory disease, a reperfusion injury, rheumatic fever, rheumatoid arthritis, sarcoidosis, a transplant rejection, and vasculitis.

4. (canceled)

5. The method according to claim 1, wherein the azol component comprises at least one compound according to Formula I or a pharmaceutically acceptable salt thereof: 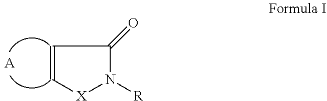
wherein X is selenium or sulfur;
wherein each R is individually selected from the group consisting of:
H;
alkyl having a carbon chain of 1 to 14 carbon atoms, wherein the carbon chain is branched or unbranched, and which is optionally substituted with one or more of: bensisoselenazol-3(2H)-one-2-yl, bensisotiazol-3(2H)-one-2-yl, OH, alkoxyl, SH, NH₂, N-alkylamino, N,N-dialkylamino wherein the alkyl groups are identical or different, COOH, aryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, I, or heteroaryl which is optionally substituted with one or more of: C₁-C₅ alkyl, OH, alkoxyl, SH, NH₂, N-alkylamino, N,N-dialkylamino, COOH, CHO, NO₂, F, Cl, Br, or I;
aryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, or I; and
heteroaryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, or I;
wherein A represents a saturated, unsaturated or polyunsaturated 3 to 6 member carbon chain, which is optionally substituted with one or more of: OR², SR², and alkylamino, C₁-C₅ alkyl, OH, alkoxyl, SH, NH₂, N-alkylamino, N,N-dialkylamino wherein the alkyl groups are identical or different, COOH, CHO, NO₂, F, Cl, Br, and I, wherein N—R¹ may optionally substitute for one or more carbons;
wherein each R¹ individually is an electron pair, H, an alkyl chain of 1-14 carbon atoms, aryl, or heteroaryl; and
wherein R² is selected from the group consisting of:
alkyl having a carbon chain of 1 to 14 carbon atoms, wherein the carbon chain is branched or unbranched, and which is optionally substituted with one or more of: bensisoselenazol-3(2H)-one-2-yl, bensisotiazol-3(2H)-one-2-yl, OH, alkoxyl, SH, NH2, N-alkylamino, N,N-dialkylamino wherein the alkyl groups are identical or different, COOH, aryl which is optionally substituted with one or more of: C1-C5 alkyl, OH, alkoxyl, SH, NH₂, N-alkylamino, N,N-dialkylamino wherein the alkyl groups are identical or different, COOH, CHO, NO₂, F, Cl, Br, I, or heteroaryl which is optionally substituted with one or more of: C₁-C₅ alkyl, OH, alkoxyl, SH, NH₂, N-alkylamino, N,N-dialkylamino, COOH, CHO, NO₂, F, Cl, Br, or I;
aryl or heteroaryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, or I; and
heteroaryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, or I.

6-16. (canceled)

17. The method of claim 16, wherein after oral administration of the integral capsule to the mammal, the glutathione component in the immediate release portion is solubilized in a stomach of the mammal and the azol component in the delayed release granule is not solubilized in the stomach of the mammal.

18. The method according to claim 1, wherein the azol component is provided within a delayed release portion comprising a surfactant and an outer coating which dissolves after passage through the stomach after oral administration, which is physically isolated from the glutathione component within a common unit dosage form.

19. (canceled)

20. The method according to claim 1, wherein the azol component is dispersed within a slowly dissolving matrix separated from the glutathione component.

21. The method according to claim 12, wherein the unit dosage form further comprises a pharmaceutically acceptable antibiotic in an amount effective to treat a bacterial infection of the mammal.

22-24. (canceled)

25. The method according to claim 5, wherein the azol component comprises the compound of Formula I in an amount of 3 μM to about 1 mM per dosage form.

26-27. (canceled)

28. A pharmaceutically acceptable unit dosage form, comprising:
glutathione in an amount of at least 250 mg, in a charge transfer complex with a sacrificial antioxidant; and
an azol comprising an isoselenazol, an isothiazol, or a pharmaceutically acceptable salt thereof, each individually in a therapeutically effective amount of at least 10 mg to treat at least one chronic inflammatory disorder of a human.

29-30. (canceled)

31. The pharmaceutically acceptable unit dosage form of claim 28, wherein the glutathione is provided within an immediate release formulation; the azol is provided within a delayed release formulation; and the glutathione within the immediate release formulation is chemically separated from the azol within the delayed release formulation.

32. The pharmaceutically acceptable unit dosage form of claim 28, wherein the azol comprises a compound according to Formula I or a pharmaceutically acceptable salt thereof: 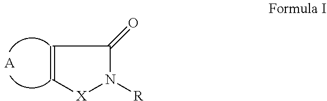
wherein X is selenium or sulfur;
wherein each R is individually selected from the group consisting of:
H;
alkyl having a carbon chain of 1 to 14 carbon atoms, wherein the carbon chain is branched or unbranched, and which is optionally substituted with one or more of: bensisoselenazol-3(2H)-one-2-yl, bensisotiazol-3(2H)-one-2-yl, OH, alkoxyl, SH, NH₂, N-alkylamino, N,N-dialkylamino wherein the alkyl groups are identical or different, COOH, aryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, I, or heteroaryl which is optionally substituted with one or more of: C₁-C₅ alkyl, OH, alkoxyl, SH, NH₂, N-alkylamino, N,N-dialkylamino, COOH, CHO, NO₂, F, Cl, Br, or I;
aryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, or I; and
heteroaryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, or I;
wherein A represents a saturated, unsaturated or polyunsaturated 3 to 6 member carbon chain, which is optionally substituted with one or more of: OR², SR², and alkylamino, C₁-C₅ alkyl, OH, alkoxyl, SH, NH₂, N-alkylamino, N,N-dialkylamino wherein the alkyl groups are identical or different, COOH, CHO, NO₂, F, Cl, Br, and I, wherein N—R¹ may optionally substitute for one or more carbons;
wherein each R¹ individually is an electron pair, H, an alkyl chain of 1-14 carbon atoms, aryl, or heteroaryl; and
wherein R² is selected from the group consisting of:
alkyl having a carbon chain of 1 to 14 carbon atoms, wherein the carbon chain is branched or unbranched, and which is optionally substituted with one or more of: bensisoselenazol-3(2H)-one-2-yl, bensisotiazol-3(2H)-one-2-yl, OH, alkoxyl, SH, NH₂, N-alkylamino, N,N-dialkylamino wherein the alkyl groups are identical or different, COOH, aryl which is optionally substituted with one or more of: C1-C5 alkyl, OH, alkoxyl, SH, NH₂, N-alkylamino, N,N-dialkylamino wherein the alkyl groups are identical or different, COOH, CHO, NO₂, F, Cl, Br, I, or heteroaryl which is optionally substituted with one or more of: C₁-C₅ alkyl, OH, alkoxyl, SH, NH₂, N-alkylamino, N,N-dialkylamino, COOH, CHO, NO₂, F, Cl, Br, or I;
aryl or heteroaryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, or I; and
heteroaryl which is optionally substituted with one or more of: C₁-C₅ alkyl, OH, alkoxyl, SH, NH₂, N-alkylamino, N,N-dialkylamino wherein the alkyl groups are identical or different, COOH, CHO, NO₂, F, Cl, Br, or I.

33. The pharmaceutically acceptable unit dosage form of claim 32, wherein in the compound of Formula I is

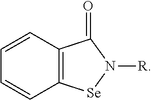

34-36. (canceled)

37. The pharmaceutically acceptable unit dosage form of claim 21, wherein the glutathione comprises reduced L-glutathione which is pharmaceutically stabilized with a charge transfer complex forming agent that serves as a sacrificial antioxidant, the glutathione being separated within the pharmaceutically acceptable unit dosage form from the azol.

38-40. (canceled)

41. The pharmaceutically acceptable unit dosage form of claim 37, wherein the dosage form is packed in a multidose pack under an inert gas.

42-47. (canceled)

48. The pharmaceutically acceptable unit dosage form of claim 28, wherein azol is provided within a delayed release portion which is physically isolated from the glutathione, the delayed release form comprising an outer coating which is configured to dissolve after passage through a stomach of a mammal after oral administration, and a surfactant to facilitate dissolution of the azol, after the outer coating is dissolved within a common unit dosage form.

49. (canceled)

50. The pharmaceutically acceptable unit dosage form of claim 28, wherein the azol is dispersed within a slowly dissolving matrix.

51. The pharmaceutically acceptable unit dosage form of claim 28, wherein the dosage form further comprises a pharmaceutically acceptable antibiotic.

52-56. (canceled)

57. A pharmaceutically acceptable unit dosage form, comprising:
at least 250 mg reduced L-glutathione;
an agent which forms a charge transfer complex with the reduced L-glutathione to substantially cancel a mixing-induced triboelectric charge on the glutathione and acts as a sacrificial antioxidant; and
at least 25 mg of an isoselenazol, or an isothiazol, which is a mammalian glutathione peroxidase mimic, and a bacterial thioredoxin reductase inhibitor.

58. The pharmaceutically acceptable unit dosage form of claim 57, wherein the unit dosage form has an immediate release portion comprising the reduced L-glutathione and the agent, and a delayed release portion comprising the isoselenazol, or the isothiazol, wherein the reduced L-glutathione of the immediate release portion is physically separated within the unit dose form from the isoselenazol or the isothiazol of the delayed release portion,
wherein the pharmaceutically acceptable unit dosage form is configured after administration by a human to: (a) substantially release the reduced L-glutathione in solution in the stomach, and (b) substantially release the isoselenazol or the isothiazol after the ligament of Treitz, to provide non-overlapping physiological release profiles.

59-88. (canceled)
